(19) United States
(12) Patent Application Publication
(10) Pub. No.: US 2019/0358613 A1
(43) Pub. Date: Nov. 28, 2019

(54) SUPPORTED INTERMETALLIC COMPOUNDS AND USE AS CATALYST

(71) Applicant: BASF SE, Ludwigshafen am Rhein (DE)

(72) Inventors: Stefan ERNST, Kaiserslautern (DE); Oliver MALTER, Kaiserslautern (DE); Axel SCHUESSLER, Kaiserslautern (DE); Kirsten BRAUNSMANN, Ludwigshafen (DE); Natalia TRUKHAN, Ludwigshafen (DE); Ulrich MUELLER, Ludwigshafen (DE)

(73) Assignee: BASF SE, Ludwigshafen am Rhein (DE)

(21) Appl. No.: 16/485,077

(22) PCT Filed: Mar. 9, 2018
(86) PCT No.: PCT/EP2018/055900

§ 371 Date: Aug. 9, 2019

(30) Foreign Application Priority Data
Mar. 10, 2017 (EP) 17160332.7

(51) Int. Cl. B01J 29/48; B01J 29/46; B01J 21/08; B01J 23/755; B01J 23/825; B01J 23/843; B01J 23/835; B01J 23/86; B01J 23/75; B01J 23/80; B01J 21/12; B01J 35/10; B01J 37/18; B01J 37/34; B01J 37/08; C22C 1/04; C07C 49/303; C07C 35/08

(52) U.S. Cl. CPC B01J 29/48; C07C 35/08; B01J 21/08; B01J 23/755; B01J 23/825; B01J 23/8435; B01J 23/835; B01J 23/864; B01J 23/75; B01J 23/80; B01J 21/12; B01J 35/1019; B01J 37/18; B01J 37/343; B01J 37/08; C22C 1/0491; C22C 1/0433; C22C 1/0425; C07C 49/303; B01J 29/46

(57) ABSTRACT

A composition comprising a ternary intermetallic compound X₂YZ, wherein X, Y, and Z are different from one another; X being selected from the group consisting of Mn, Fe, Co, Ni, Cu, and Pd; Y being selected from the group consisting of Cr, Co, and Ni; and Z being selected from the group consisting of Al, Si, Ga, Ge, In, Sn, Zn, and Sb; wherein the ternary intermetallic compound is supported on a porous oxidic support material. The composition may be prepared by providing a liquid mixture of sources of X, Y, and Z, and the porous oxidic support material, removing the liquid and heating the resulting mixture in a reducing atmosphere. The composition is useful as catalyst.

The present invention relates to a composition comprising a ternary intermetallic compound X₂YZ supported on a support material, wherein X, Y, and Z are different from one another. Further, the present invention relates to a process for preparing said ternary intermetallic compound. Yet further, the present invention relates to the use of said ternary intermetallic compound.

Heusler phases are intermetallic compounds with X₂YZ composition wherein X and Y are transition metals and Z is a third/fourth row main group element. Since their discovery, the main interest for said compounds mainly focused on ferromagnetic applications such as in spintronics, thermoelectrics, and giant magnetoresistance. In particular, their catalytic properties were barely touched such as in Hedin et al. which is a study on how changes in ferromagnetism may influence catalytic reactions such as the hydrogenation of carbon monoxide and ethylene over nickel and the oxidation of carbon monoxide to carbon dioxide over the Heusler alloy MnAlCu₂. Kojima et al. disclose the catalytic properties of specific Heusler phases. Therefore, there remains a need for new ternary intermetallic compounds having the X₂YZ composition which can be used in particular in various fields of catalysis. Accordingly, it was an object of the present invention to provide new ternary intermetallic compounds having the X₂YZ composition which can be used in catalytic reactions.

Senanayake et al. “Exploring Heusler alloys as catalysts for ammonia dissociation”, August 2016, ISBN: 978-1-369-00770-1, discloses activation energy of ammonia cracking on the surfaces of various compositions of Heusler alloys, like NiMnGa and CoCrGe.

Okamura et al. “Structural, magnetic, and transport properties of full-Heusler alloy Co₂(Cr₁₋ₓ Feₓ)Al thin films” J. Appl. Phys. vol. 96, no. 11, 1 Dec. 2004, pages 6561-6564, discloses the structural, magnetic, and transport properties of full-Heusler alloy Co₂(Cr₁₋ₓFeₓ)Al thin films sputtered on thermally oxidized Si substrates at room temperature.

Kelekar et al. “Epitaxial growth of the Heusler alloy Co₂Crₐ₋ₓFeₓAl” J. Appl. Phys. Vol. 96, no 1, 1 Jul. 2004, pages 540-543, discloses a method for the growth of single-phase epitaxial thin films of compounds from the family of Heusler alloys Co₂Cr₁₋ₓFeₓAl.

Ko et al. “Half-metallic Fe₂CrSi and non-metallic Cu₂CrAl Heusler alloys for currentperpendicular-to-plane giant magneto-resistance: First principle and experimental study” J. Appl. Phys. Vol 109, no. 7, 17 Mar. 2011, pages 7B1031-7B1033, discloses Fe—Cr—Si and CuCr—Al films on Cr-buffered MgO substrates.

Therefore, the present invention relates to a composition comprising a ternary intermetallic compound X₂YZ, wherein

X, Y, and Z are different from one another;

X being selected from the group consisting of Mn, Fe, Co, Ni, Cu, and Pd;

Y being selected from the group consisting of Cr, Co, and Ni; and

Z being selected from the group consisting of Al, Si, Ga, Ge, In, Sn, Zn, and Sb; wherein the ternary intermetallic compound is supported on a porous oxidic support material.

The term “X₂YZ” as used in the present invention refers to compositions having a composition XₐY_{b}Z_{c} wherein a is in the range of from 1.9 to 2.1 such as in the range of from 1.90 to 2.05 or from 1.95 to 2.10 or from 1.95 to 2.05; wherein b is in the range of from 0.9 to 1.1 such as in the range of from 0.90 to 1.05 or from 0.95 to 1.10 or from 0.95 to 1.05; and wherein c is in the range of from 0.9 to 1.1 such as in the range of from 0.90 to 1.05 or from 0.95 to 1.10 or from 0.95 to 1.05.

Generally, any conceivable porous oxidic support material can be used. Preferably, the porous oxidic support material comprises one or more of silica, alumina, titania, zirconia, a mixed oxide of one or more Si, Al, Ti, and Zr, and a mixture of two or more thereof. Preferably, at least 99 weight-%, more preferably at least 99.5 weight-%, more preferably at least 99.9 weight-% of the porous oxidic support material consist of one or more of silica, alumina, titania, zirconia, a mixed oxide of one or more Si, Al, Ti, and Zr, and a mixture of two or more thereof.

According to a preferred embodiment of the present invention, the intermetallic compound comprises Co. Therefore, preferably either X or Y is Co. While generally all respective combinations of X, Y, and Z are conceivable, it is preferred that if X is Co, Y is Cr and, if Y is Co X is Cu. In particular for these combinations of X and Y, it is preferred that Z is selected from the group consisting of Al, Ga, In, and Zn.

Therefore, the ternary intermetallic compound is preferably selected from the group consisting of Co₂CrAl, Co₂CrIn, Co₂CrZn, Co₂CrGa, Cu₂CoAl, Cu₂CoIn, Cu₂CoZn, and Cu₂CoGa. More preferably, the ternary intermetallic compound is selected from the group consisting of Co₂CrAl, Co₂CrIn, Co₂CrZn, Co₂CrGa, Cu₂CoAl, Cu₂CoZn, and Cu₂CoGa.

With regard to said Co-based intermetallic compounds, the porous oxidic support material preferably comprises Si. More preferably, the porous oxidic support material comprises silica or a mixed oxide of Si and Al. More preferably, the porous oxidic support material comprises a mixed oxide of Si and Al. Preferably at least 99 weight-%, more preferably at least 99.5 weight-%, more preferably at least 99.9 weight-% of the porous oxidic support material consist of the mixed oxide of Si and Al.

Generally, every porous mixed oxide of Si and Al can be employed. Preferably, porous mixed oxide of Si and Al is a zeolitic material. Zeolites are microporous, aluminosilicate minerals, occur naturally and are also produced industrially, in some instances on a large scale. Zeolites are the aluminosilicate members of the family of microporous solids known as “molecular sieves” mainly consisting of Si, Al, O. A microporous material is a material containing pores with diameters less than 2 nm. Preferably, the zeolitic material has a framework type which is ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEl, EMT, EON, EPI, ERI, ESV, ETL, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SVV, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, a mixture of two or more of these framework types, or a mixed framework type thereof. Such three letter code abbreviations and the respective explanations can be found, for example, under “www.iza-structure.org”, in section “Framework Type”, or in “Atlas of Zeolite Framework Types, 6th revised edition, Elsevier, 2007”, accessible online via “http://www.iza-structure.org/databases/books/Atlas_6ed.pdf”. More preferably, the zeolitic material comprises framework type MFI. More preferably, the zeolitic material has framework type MFI. More preferably, the zeolitic material comprises a zeolite ZSM-5. More preferably, the zeolitic material is a zeolite ZSM-5.

Therefore, the present invention preferably relates to a composition comprising a ternary intermetallic compound X₂YZ, wherein the ternary intermetallic compound is selected from the group consisting of Co₂CrAl, Co₂CrIn, Co₂CrZn, Co₂CrGa, Cu₂CoAl, Cu₂CoIn, Cu₂CoZn, and Cu₂CoGa, preferably from the group consisting of Co₂CrAl, Co₂CrIn, Co₂CrZn, Co₂CrGa, Cu₂CoAl, Cu₂CoZn, and Cu₂CoGa, wherein the ternary intermetallic compound is supported on a zeolitic material preferably having framework type MFI, more preferably being a zeolite ZSM-5.

The loading of the porous oxidic support materials with the Co-based ternary intermetallic compound X₂YZ is not subject to any specific restrictions. Preferably, in the composition of the present invention, the weight ratio of the ternary intermetallic compound relative to the porous oxidic compound is in the range of from 0.5:99.5 to 30:70, preferably in the range of from 1:99 to 20:80, more preferably in the range of from 2:99 to 10:90, more preferably in the range of from 3:97 to 7:93. Preferably, the present invention relates to a composition wherein the ternary intermetallic compound is selected from the group consisting of Co₂CrAl, Co₂CrIn, Co₂CrZn, Co₂CrGa, Cu₂CoAl, Cu₂CoIn, Cu₂CoZn, and Cu₂CoGa, preferably from the group consisting of Co₂CrAl, Co₂CrIn, Co₂CrZn, Co₂CrGa, Cu₂CoAl, Cu₂CoZn, and Cu₂CoGa, wherein in the composition, the weight ratio of the ternary intermetallic compound relative to the porous oxidic compound is in the range of from 3:97 to 7:93, preferably in the range of from 4:96 to 6:94. More preferably, the present invention relates to a composition wherein the ternary intermetallic compound is selected from the group consisting of Co₂CrAl, Co₂CrIn, Co₂CrZn, Co₂CrGa, Cu₂CoAl, Cu₂CoIn, Cu₂CoZn, and Cu₂CoGa, preferably from the group consisting of Co₂CrAl, Co₂CrIn, Co₂CrZn, Co₂CrGa, Cu₂CoAl, Cu₂CoZn, and Cu₂CoGa, wherein in the composition, the weight ratio of the ternary intermetallic compound relative to the porous oxidic compound is in the range of from 3:97 to 7:93, preferably in the range of from 4:96 to 6:94, wherein the ternary intermetallic compound is supported on a zeolitic material preferably having framework type MFI, more preferably being a zeolite ZSM-5.

According to a preferred embodiment of the present invention, Y is Ni. In this regard, it is preferred that X is Cu. Therefore, preferred compositions of the present comprise Cu as X and Ni as Y. Further in this regard, it is preferred that Z is Al, Si, Ga, In, Sn, or Sb. More preferably, Z is Sn. Therefore, preferred compositions of the present comprise Al, Si, Ga, In, Sn, or Sb as Z, more preferably Sn as Z, and Ni as Y. More preferably, the composition of the present invention comprises a ternary intermetallic compound which is Cu₂NiSn.

With regard to said Ni-based intermetallic compounds, the porous oxidic support material preferably comprises Si. More preferably, the porous oxidic support material comprises silica or a mixed oxide of Si and Al. With regard to preferred mixed oxides of Si and Al and in particular preferred zeolitic materials, reference is made to the disclosure above. More preferably, the porous oxidic support material comprises silica. Preferably at least 99 weight-%, more preferably at least 99.5 weight-%, more preferably at least 99.9 weight-% of the porous oxidic support material consist of silica.

The loading of the porous oxidic support materials with the Ni-based ternary intermetallic compound X₂YZ is not subject to any specific restrictions. Preferably, in the composition of the present invention, the weight ratio of the ternary intermetallic compound relative to the porous oxidic support is in the range of from 1:99.5 to 70:30, preferably in the range of from 5:99 to 60:40, more preferably in the range of from 10:90 to 50:50, more preferably in the range of from 10:90 to 45:55, more preferably in the range of from 10:90 to 40:60.

Preferably, the present invention relates to a composition wherein the ternary intermetallic compound is Cu₂NiSn, wherein in the composition, the weight ratio of the ternary intermetallic compound relative to the porous oxidic compound is in the range of from 20:80 to 40:60, preferably in the range of from 25:75 to 35:65, preferably in the range of from 28:72 to 32:68. More preferably, the present invention relates to a composition wherein the ternary intermetallic compound is Cu₂NiAl, wherein in the composition, the weight ratio of the ternary intermetallic compound relative to the porous oxidic compound is in the range of from 20:80 to 40:60, preferably in the range of from 25:75 to 35:65, preferably in the range of from 28:72 to 32:68, wherein the ternary intermetallic compound is supported on a porous oxidic support material which comprises, preferably is silica.

Within the meaning of the present invention, the terms “D10”, “D50”, and “D90” respectively refer to the particle size by number of the particles, formed by the ternary intermetallic compound of the present invention, wherein D10 refers to the particle size wherein 10% of the particles, formed by the ternary intermetallic compound, by number lie below said value, D50 refers to the particle size wherein 50% of the particles, formed by the ternary intermetallic compound, by number lie below said value, and D90 accordingly refers to the particle size wherein 90% of the particles, formed by the ternary intermetallic compound, by number lie below said particle size.

The mean particle size “D50” as well as the particle sizes “D90” and “D10” as used herein may readily be measured by known methods, wherein preferably they are determined by Transmission Electron Microscopy (TEM), preferably wherein the samples for TEM, preferably a powder, are prepared on ultra-thin carbon TEM carriers, preferably by dispersing the powder in ethanol, preferably by applying one drop of the dispersion between two glass objective slides which is then dispersed, preferably wherein the TEM carrier film is then subsequently dipped on the resulting thin film, wherein more preferably the TEM images are recorded on a Tecnai Osiris machine operated at 200 keV under bright-field as well as high-angle annular dark-field scanning TEM (HAADF-STEM) conditions. Preferably Chemical composition maps are acquired by energy-dispersive x-ray spectroscopy (EDXS), wherein more preferably images and elemental maps are evaluated using the iTEM as well as the Esprit software packages. Preferably, the particle size distributions are evaluated using the ParticleSizer plugin for FIJI. According to the present invention it is more preferred that the mean particle size D50 as well as the particle sizes D90 and D10 as used herein are determined according to the method described herein under the examples, more preferably as described in reference example 1.1.

Preferably, the Ni-based composition of the invention comprises particles, formed by the ternary intermetallic compound, having a particle size, determined via TEM as described in Reference Example 1.1 herein, in the range of from 0.1 nm to 2 micrometer, preferably of from 0.5 nm to 2 micrometer, more preferably of from 1 nm to 2 micrometer, and more preferably of from 2 nm to 2 micrometer, wherein at least 10 weight-%, preferably from 10 to 30 weight-% of the composition consist of these particles.

Preferably, the Ni-based composition of the invention comprises particles, formed by the ternary intermetallic compound, having a particle size D10 in the range of from 0.5 to 10 nm, preferably from 1 to 9 nm, more preferably from 2 to 8 nm, more preferably from 3 to 7 nm, and more preferably from 4 to 6 nm.

Preferably, the Ni-based composition of the invention comprises particles, formed by the ternary intermetallic compound, having a particle size D50 in the range of from 1 to 13 nm, preferably from 2 to 12 nm, more preferably from 3 to 11 nm, more preferably from 4 to 10 nm, more preferably from 5 to 9 nm, and more preferably from 6 to 8 nm.

Preferably, the Ni-based composition of the invention comprises particles, formed by the ternary intermetallic compound, having a particle size D90 in the range of from 7 to 19 nm, preferably from 8 to 18 nm, more preferably from 9 to 17 nm, more preferably from 10 to 16 nm, more preferably from 11 to 15 nm, and more preferably from 12 to 14 nm.

Preferably, the Ni-based composition of the invention comprises particles, formed by the ternary intermetallic compound, having a particle size D10 in the range of from 2 to 8 nm, preferably from 3 to 7 nm, and more preferably from 4 to 6 nm;

wherein the particle size D50 is in the range of from 4 to 10 nm, preferably from 5 to 9 nm, and more preferably from 6 to 8 nm; and

wherein the particle size D90 in the range of from 10 to 16 nm, preferably from 11 to 15 nm, and more preferably from 12 to 14 nm.

It is alternatively preferred that the Ni-based composition of the invention comprises particles, formed by the ternary intermetallic compound, having a particle size D10 in the range of from 35 to 59, preferably from 37 to 57 nm, more preferably from 39 to 55 nm, more preferably from 41 to 53 nm, more preferably from 43 to 51 nm, and more preferably from 45 to 49 nm.

It is alternatively preferred that the Ni-based composition of the invention comprises particles, formed by the ternary intermetallic compound, having a particle size D50 in the range of from 55 to 79 nm, preferably from 57 to 77 nm, more preferably from 59 to 75 nm, more preferably from 61 to 73 nm, more preferably from 63 to 71 nm, and more preferably from 65 to 69 nm.

It is alternatively preferred that the Ni-based composition of the invention comprises particles, formed by the ternary intermetallic compound, having a particle size D90 in the range of from 108 to 152 nm, preferably from 112 to 148 nm, more preferably from 116 to 144 nm, more preferably from 120 to 140 nm, more preferably from 124 to 136 nm, and more preferably from 128 to 132 nm.

It is alternatively preferred that the Ni-based composition of the invention comprises particles, formed by the ternary intermetallic compound, having a particle size D10 in the range of from 41 to 53 nm, preferably from 43 to 51 nm, and more preferably from 45 to 49 nm;

wherein the particle size D50 is in the range of from 61 to 73 nm, preferably from 63 to 71 nm, and more preferably from 65 to 69 nm; and

wherein the particle size D90 in the range of from 120 to 140 nm, preferably from 124 to 136 nm, and more preferably from 128 to 132 nm.

Preferably, in the Ni-based composition of the invention, the crystallite size, determined via XRD using the Scherer equation as described in Reference Example 1.2 herein, is in the range of from 8 to 30 nm. Preferably, the Ni-based composition of the invention has a BET specific surface area, determined as described in Reference Example 1.3 herein, in the range of from 150 to 400 m²/g.

Generally, the composition of the present invention may comprise, in addition to the ternary intermetallic compound and the porous oxidic support material, one or more further compounds.

Preferably, the composition of the present invention essentially consists of the ternary intermetallic compound and the porous oxidic support material. Therefore, preferably at least 99 weight %, more preferably at least 99.5 weight-%, more preferably at least 99.9 weight-% of the composition consist of the ternary intermetallic compound and the porous oxidic support material.

Among others, it is preferred that the intermetallic compound of the composition of the present invention is a Heusler phase.

Preferably, the intermetallic compound is supported on the porous oxidic material in the form of particles.

Generally, the composition of the present invention can be prepared by any suitable process.

Preferably, it is prepared by a process comprising
(i) preparing a liquid mixture comprising a source of X, a source of Y, a source of Z, and a source of the porous oxidic support material;
(ii) removing the liquid phase from the mixture prepared in (i);
(iii) heating the mixture obtained from (ii) in a reducing atmosphere, obtaining the intermetallic compound supported on a porous oxidic support material.

Preferably, the source of X is selected from the group consisting of salts of X. Said salts of X are preferably selected from the group consisting of acetates, acetylacetonates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof. More preferably, said salts are selected from the group consisting of acetates, acetylacetonates, nitrates, chlorides, bromides, fluorides, and mixtures of two or more thereof. More preferably, said salts are selected from the group consisting of acetates, acetylacetonates, nitrates and chlorides.

Preferably, the source of Y is selected from the group consisting of salts of Y. Said salts of Y are preferably selected from the group consisting of acetates, acetylacetonates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof. More preferably, said salts are selected from the group consisting of acetates, acetylacetonates, nitrates, chlorides, bromides, fluorides, and mixtures of two or more thereof. More preferably, said salts are selected from the group consisting of acetates, acetylacetonates, and nitrates.

Preferably, the source of Z is selected from the group consisting of salts of Z. Said salts of Z are preferably selected from the group consisting of C1-C4 alkoxides, acetates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof. More preferably, said salts are selected from the group consisting of C₂-C₃ alkoxides, acetates, nitrates, chlorides, bromides, fluorides, and mixtures of two or more thereof. More preferably, said salts are selected from the group consisting of ethoxides, acetates, nitrates, and chlorides.

Therefore, it is preferred that the source of X is an acetate, an acetylacetonate, a nitrate or a chloride of X, the source of Y is an acetate, an acetylacetonates, or a nitrate of Y, and the source of Z is an ethoxide, an acetates, a nitrates or a chloride of Z.

Preferably, the source of the porous oxidic support material comprises one or more of silica, alumina, titania, zirconia, a mixed oxide of one or more Si, Al, Ti, and Zr, and a mixture of two or more thereof. Preferably at least 99 weight-%, more preferably at least 99.5 weight-%, more preferably at least 99.9 weight-% of the porous oxidic support material consist of one or more of silica, alumina, titania, zirconia, a mixed oxide of one or more Si, Al, Ti, and Zr, and a mixture of two or more thereof.

According to a preferred embodiment of the present invention according to which the ternary intermetallic compound is a Co-based compound, the source of the porous oxidic support material comprises silica or a mixed oxide of Si and Al, preferably a mixed oxide of Si and Al. More preferably, at least 99 weight-%, more preferably at least 99.5 weight-%, more preferably at least 99.9 weight-% of the porous oxidic support material consist of the mixed oxide of Si and Al.

Generally, every porous mixed oxide of Si and Al can be employed as source of the porous oxidic support material. Preferably, porous mixed oxide of Si and Al is a zeolitic material. Zeolites are microporous, aluminosilicate minerals, occur naturally and are also produced industrially, in some instances on a large scale. Zeolites are the aluminosilicate members of the family of microporous solids known as “molecular sieves” mainly consisting of Si, Al, O. A microporous material is a material containing pores with diameters less than 2 nm. Preferably, the zeolitic material has a framework type which is ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEl, EMT, EON, EPI, ERI, ESV, ETL, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SVV, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, a mixture of two or more of these framework types, or a mixed framework type thereof. Such three letter code abbreviations and the respective explanations can be found, for example, under “www.izastructure.org”, in section “Framework Type”, or in “Atlas of Zeolite Framework Types, 6th revised edition, Elsevier, 2007”, accessible online via “http://www.izastructure.org/databases/books/Atlas_6ed.pdf”. More preferably, the zeolitic material comprises framework type MFI. More preferably, the zeolitic material has framework type MFI. More preferably, the zeolitic material comprises a zeolite ZSM-5. More preferably, the zeolitic material is a zeolite ZSM-5.

Therefore, the present preferably relates to the process described above, wherein the source of X is an acetate, an acetylacetonate, a nitrate or a chloride of X, the source of Y is an acetate, an acetylacetonates, or a nitrate of Y, and the source of Z is an ethoxide, an acetates, a nitrates or a chloride of Z, and the source of the porous oxidic compound is a zeolitic material preferably having framework type MFI, more preferably being a zeolite ZSM-5.

According to a preferred embodiment of the present invention according to which the ternary intermetallic compound is a Ni-based compound, it is preferred that the source of the porous oxidic compound comprises silica or a mixed oxide of Si and Al, preferably silica. More preferably, at least 99 weight-%, more preferably at least 99.5 weight-%, more preferably at least 99.9 weight-% of the porous oxidic support material consist of silica.

Generally, any suitable silica can be employed, including both colloidal silica and so-called “wet process” silica and so-called “dry process” silica can be used. More preferably, the silica is amorphous silica. Colloidal silica, preferably as an alkaline and/or ammoniacal solution, more preferably as an ammoniacal solution, is commercially available, inter alia, for example as Ludox®, Syton®, Nalco® or Snowtex®. “Wet process” silica is commercially available, inter alia, for example as Hi-Sil®, Ultrasil®, Vulcasil®, Santocel®, Valron-Estersil®, Tokusil® or Nipsil®. “Dry process” silica is commercially available, inter alia, for example as Aerosil®, Reolosil®, Cab-O-Sil®, Fransil® or ArcSilica®. Inter alia, an ammoniacal solution of colloidal silica can be used according to the present invention. More preferably, the silica comprises, preferably is fumed silica. Preferably, the silica has a BET specific surface area, determined as described in Reference Example 1.3, in the range of from 300 to 500 m²/g, more preferably in the range of from 350 to 450 m²/g. Preferably, the silica has a total pore volume, determined as described in Reference Example 1.3, in the range of from 0.4 to 0.5 ml/g, more preferably in the range of from 0.42 to 0.48 ml/g. Preferably, the silica has an average pore size, determined as described in Reference Example 1.3, in the range of from 4 to 5 nm, more preferably in the range of from 4.2 to 4.8 nm.

Preferably, step (i) of the process of the present invention comprises
(i.1) preparing a liquid mixture comprising a source of X, a source of Y, a source of Z, and a solvent;
(i.2) admixing the source of the porous oxidic support material with the mixture prepared in (i.1).

As solvent according to (i.1), it is preferred to employ a polar solvent, more preferably one or more polar protic solvents. More preferably, the one or more solvents are selected from the group consisting of water, C₁ alcohols, C₂ alcohols, C₃ alcohols, C₄ alcohols, and mixtures of two or more thereof, more preferably selected from the group consisting of water, C₁ alcohols, C₂ alcohols, C₃ alcohols, and mixtures of two or more thereof. More preferably, the solvent is one or more of water, methanol and ethanol, wherein more preferably, the solvent according to (i.) comprises, preferably is, methanol.

According to (i.2), it is preferred to prepare a liquid mixture comprising a solvent and the source of the porous oxidic support material and admixing the liquid mixture with the mixture prepared in (i.1). As solvent according to (i.2), it is preferred to employ a polar solvent, more preferably one or more polar protic solvents. More preferably, the one or more solvents are selected from the group consisting of water, C alcohols, C₂ alcohols, C₃ alcohols, C₄ alcohols, and mixtures of two or more thereof, more preferably selected from the group consisting of water, C₁ alcohols, C₂ alcohols, C₃ alcohols, and mixtures of two or more thereof. More preferably, the solvent is one or more of water, methanol and ethanol, wherein more preferably, the solvent according to (i.2) comprises, preferably is, methanol. Preferably, the solvent according to (i.2) is the solvent according to (i.1).

According to (ii), the liquid phase is removed from the mixture. Generally, this can be accomplished by every suitable method or combination of methods. According to the present invention, it is preferred to remove the liquid phase either by suitably heating the mixture, or by suitably subjecting the mixture to evaporation, or by suitably heating the mixture and by suitably subjecting the mixture to evaporation. If heating and evaporation are carried out, it is possible to subject the mixture to evaporation and subsequently subject to respectively obtained mixture to heating. Further, it is possible to subject the mixture to heating and subsequently subject to respectively obtained mixture to evaporation. Yet further, it is possible that evaporation and heating are carried out at least partially simultaneously.

Therefore, it is preferred that removing the liquid phase from the mixture according to (ii) comprises heating the mixture prepared in (i), preferably heating to a temperature of the mixture in the range of from 70 to 150° C., preferably in the range of from 80 to 140° C., more preferably from 90 to 130° C., more preferably from 100 to 120° C. Further, it is preferred that the mixture prepared in (i) is subjected to evaporation, preferably at a pressure in the range of from 2 to 500 mbar(abs), more preferably in the range of from 5 to 200 mbar(abs), more preferably in the range of from 10 to 100 mbar(abs). It is more preferred that according to (ii) and prior to heating, the mixture prepared in (i) is subjected to evaporation, preferably at a pressure in the range of from 2 to 500 mbar(abs), more preferably in the range of from 5 to 200 mbar(abs), more preferably in the range of from 10 to 100 mbar(abs). During evaporation, it is preferred to adjust the temperature of the mixture to a value in the range of from 20 to 60° C., preferably in the range of from 30 to 50° C.

With regard to the reducing according to (iii), it is preferred that the reducing atmosphere according to (iii) comprises hydrogen) preferably comprises hydrogen and an inert gas, such as argon or nitrogen, preferably nitrogen. Preferably in the reducing atmosphere, the volume ratio of hydrogen relative to the inert gas, preferably nitrogen, is in the range of from 30:70 to 70:30, preferably in the range of from 40:60 to 60:40.

According to (iii), it is preferred to heat the mixture to a temperature of the reducing atmosphere in the range of from 400 to 1,100° C., preferably in the range of from 500 to 1,000° C. More preferably, according to (iii), the mixture is heated to a temperature of the reducing atmosphere in the range of from 800 to 1,000° C., more preferably in the range of from 850 to 1000° C.

Generally, the mixture can be heated to said temperature using any suitable temperature ramp or heating rates. Preferably, according to (iii), the mixture is heated at a temperature ramp in the range of from 0.1 to 15 K/min, preferably in the range of from 0.3 to 12 K/min. According to a first embodiment, the mixture is heated at a temperature ramp preferably in the range of from 0.4 to 7 K/min, more preferably in the range of from 0.5 to 5 K/min. According to a second embodiment, the mixture is heated at a temperature ramp preferably in the range of from 8 to 12 K/min, preferably in the range of from 9 to 11 K/min. During heating, the temperature ramp can be varied; for example, the mixture can be heated at a first temperature ramp to a first temperature, heated at a second temperature ramp to a second temperature, optionally heated at a third temperature ramp to a third temperature, wherein at least one of the first, second and third temperature ramp is different from at least of the other temperature ramps. Further, it is possible that, once the first or the second temperature is reached, the mixture is kept at this temperature for a certain period of time. Once the desired maximum temperature as described above is reached, it is preferred to keep the mixture at the temperature for a period of time in the range of from 0.5 to 20 h, preferably in the range of from 1 to 15 h, more preferably in the range of from 2 to 10 h.

After said heat treatment, it is preferred to cool the heat-treated mixture, preferably to a temperature in the range of from 10 to 50° C., more preferably in the range of from 20 to 30° C. Therefore, the process of the present invention preferably further comprises
(iv) cooling the intermetallic compound supported on a porous oxidic material, obtained from (iii).

Yet further, the present invention relates to the composition as described above, which is obtainable or obtained or preparable or prepared by a process as described above, preferably comprising steps (i) to (iiii), more preferably steps (i) to (iv).

Still further, the present invention relates to the use of the composition of the present invention as a catalytically active material, preferably for an oxidation reaction, a hydrogenation reaction, a dehydrogenation reaction, and/or a condensation reaction. Also, the present invention relates to a method for catalytically converting an organic compound, comprising bringing the organic compound in contact with a catalyst which comprises the composition of the present invention as a catalytically active material, wherein the converting of the organic compound comprises an oxidation reaction, a hydrogenation reaction, a dehydrogenation reaction, and/or a condensation reaction. Preferably, the hydrogenation reaction comprises the hydrogenation of an aldehyde, preferably cinnamaldehyde. Preferably, the dehydrogenation reaction comprises the dehydrogenation of an alkane, preferably propane. Preferably, the oxidation reaction comprises the oxidation of an alkane, preferably a cyclic alkane, more preferably cyclohexane. Preferably, the condensation reaction comprises the condensation of a carbonyl compound with a methylene group containing compound, wherein the condensation reaction is preferably a Knoevenagel condensation reaction.

Generally, the composition of the present invention may be used as such as a catalyst. Further, it is possible that in addition to the composition of the invention, the catalyst may comprise one or more further catalytically active materials and/or one or more inert materials including, but not restricted to, one or more matrix materials, for example one or more binder materials.

The present invention is further illustrated by the following set of embodiments and combinations of embodiments resulting from the dependencies and back-references as indicated. In particular, it is noted that in each instance where a range of embodiments is mentioned, for example in the context of a term such as “The catalyst of any one of embodiments 1 to 4”, every embodiment in this range is meant to be explicitly disclosed for the skilled person, i.e. the wording of this term is to be understood by the skilled person as being synonymous to “The catalyst of any one of embodiments 1, 2, 3, and 4”.
1. A composition comprising a ternary intermetallic compound X₂YZ, wherein
   X, Y, and Z are different from one another;
   X being selected from the group consisting of Mn, Fe, Co, Ni, Cu, and Pd;
   Y being selected from the group consisting of Cr, Co, and Ni; and
   Z being selected from the group consisting of Al, Si, Ga, Ge, In, Sn, Zn, and Sb;
   wherein the ternary intermetallic compound is supported on a porous oxidic support material.
2. The composition of embodiment 1, wherein the porous oxidic support material comprises one or more of silica, alumina, titania, zirconia, a mixed oxide of one or more Si, Al, Ti, and Zr, and a mixture of two or more thereof.
3. The composition of embodiment 1 or 2, wherein at least 99 weight-%, preferably at least 99.5 weight-%, more preferably at least 99.9 weight-% of the porous oxidic support material consist of one or more of silica, alumina, titania, zirconia, a mixed oxide of one or more Si, Al, Ti, and Zr, and a mixture of two or more thereof.
4. The composition of any one of embodiments 1 to 3, wherein X or Y is Co.
5. The composition of any one of embodiments 1 to 4, wherein X is Co and Y is Cr.
6. The composition of any one of embodiments 1 to 5, wherein Y is Co and X is Cu.
7. The composition of any one of embodiments 1 to 6, wherein Z is selected from the group consisting of Al, Ga, In, and Zn.
8. The composition of any one of embodiments 1 to 7, wherein the ternary intermetallic compound is selected from the group consisting of Co₂CrAl, Co₂CrIn, Co₂CrZn, Co₂CrGa, Cu₂CoAl, Cu₂CoIn, Cu₂CoZn, and Cu₂CoGa.
9. The composition of any one of embodiments 1 to 8, wherein the ternary intermetallic compound is selected from the group consisting of Co₂CrAl, Co₂CrIn, Co₂CrZn, Co₂CrGa, Cu₂CoAl, Cu₂CoZn, and Cu₂CoGa.
10. The composition of any one of embodiments 1 to 9, wherein ternary intermetallic compound is Co₂CrAl.
11. The composition of any one of embodiments 1 to 9, wherein ternary intermetallic compound is Cu₂CoZn.
12. The composition of any one of embodiments 1 to 11, wherein the porous oxidic support material comprises a mixed oxide of Si and Al.
13. The composition of embodiment 12, wherein at least 99 weight-%, preferably at least 99.5 weight-%, more preferably at least 99.9 weight-% of the porous oxidic support material consist of the mixed oxide of Si and Al.
14. The composition of embodiment 12 or 13, wherein the mixed oxide of Si and Al is a zeolitic material.
15. The composition of embodiment 14, wherein the zeolitic material has a framework type which is ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEl, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SVV, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, a mixture of two or more of these framework types, or a mixed framework type thereof.
16. The composition of embodiment 14 or 15, wherein the zeolitic material has framework type MFI.
17. The composition of any one of embodiments 14 to 16, wherein the zeolitic material comprises, preferably is a zeolite ZSM-5.
18. The composition of any one of embodiments 1 to 17, wherein in the composition, the weight ratio of the ternary intermetallic compound relative to the porous oxidic compound is in the range of from 0.5:99.5 to 30:70, preferably in the range of from 1:99 to 20:80, more preferably in the range of from 2:99 to 10:90, more preferably in the range of from 3:97 to 7:93.
19. The composition of any one of embodiments 1 to 18, wherein the ternary intermetallic compound is selected from the group consisting of Co₂CrAl, Co₂CrIn, Co₂CrZn, Co₂CrGa, Cu₂CoAl, Cu₂CoZn, and Cu₂CoGa, preferably Co₂CrAl or Cu₂CoZn, wherein the porous oxidic support material is a zeolitic material having framework type MFI, preferably is a zeolite ZSM-5, and wherein in the composition, the weight ratio of the ternary intermetallic compound relative to the porous oxidic compound is in the range of from 3:97 to 7:93, preferably in the range of from 4:96 to 6:94.
20. The composition of any one of embodiments 1 to 3, wherein Y is Ni.
21. The composition of embodiment 20, wherein X is Cu.
22. The composition of embodiment 20 or 21, wherein Z is Al, Si, Ga, In, Sn, or Sb, preferably Sn.
23. The composition of any one of embodiments 20 to 21, wherein the ternary intermetallic compound is Cu₂NiSn.
24. The composition of any one of embodiments 20 to 23, wherein the porous oxidic support material comprises silica.
25. The composition of embodiment 25, wherein at least 99 weight-%, preferably at least 99.5 weight-%, more preferably at least 99.9 weight-% of the porous oxidic support material consist of silica.
26. The composition of any one of embodiments 20 to 25, wherein in the composition, the weight ratio of the ternary intermetallic compound relative to the porous oxidic support is in the range of from 1:99.5 to 70:30, preferably in the range of from 5:99 to 60:40, more preferably in the range of from 10:90 to 50:50, more preferably in the range of from 10:90 to 45:55, more preferably in the range of from 10:90 to 40:60.
27. The composition of any one of embodiments 20 to 26, wherein the ternary intermetallic compound is Cu₂NiSn, wherein the porous oxidic support material is silica, and wherein in the composition, the weight ratio of the ternary intermetallic compound relative to the porous oxidic compound is in the range of from 20:80 to 40:60, preferably in the range of from 25:75 to 35:65, preferably in the range of from 28:72 to 32:68.
28. The composition of any one of embodiments 20 to 27, wherein the composition comprises particles, formed by the ternary intermetallic compound, having a particle size, determined via TEM as described in Reference Example 1.1 herein, in the range of from 0.1 nm to 2 micrometer, preferably of from 0.5 nm to 2 micrometer, more preferably of from 1 nm to 2 micrometer, and more preferably of from 2 nm to 2 micrometer, wherein at least 10 weight-%, preferably from 10 to 30 weight-% of the composition consist of these particles.
29. The composition of any one of embodiments 20 to 28, wherein the composition comprises particles, formed by the ternary intermetallic compound, having a particle size D10 in the range of from 0.5 to 10 nm, preferably from 1 to 9 nm, more preferably from 2 to 8 nm, more preferably from 3 to 7 nm, and more preferably from 4 to 6 nm.
30. The composition of any one of embodiments 20 to 29, wherein the composition comprises particles, formed by the ternary intermetallic compound, having a particle size D50 in the range of from 1 to 13 nm, preferably from 2 to 12 nm, more preferably from 3 to 11 nm, more preferably from 4 to 10 nm, more preferably from 5 to 9 nm, and more preferably from 6 to 8 nm.
31. The composition of any one of embodiments 20 to 30, wherein the composition comprises particles, formed by the ternary intermetallic compound, having a particle size D90 in the range of from 7 to 19 nm, preferably from 8 to 18 nm, more preferably from 9 to 17 nm, more preferably from 10 to 16 nm, more preferably from 11 to 15 nm, and more preferably from 12 to 14 nm.
32. The composition of any one of embodiments 20 to 28, wherein the composition comprises particles, formed by the ternary intermetallic compound, having a particle size D10 in the range of from 2 to 8 nm, preferably from 3 to 7 nm, and more preferably from 4 to 6 nm; wherein the particle size D50 is in the range of from 4 to 10 nm, preferably from 5 to 9 nm, and more preferably from 6 to 8 nm; and wherein the particle size D90 in the range of from 10 to 16 nm, preferably from 11 to 15 nm, and more preferably from 12 to 14 nm.
33. The composition of any one of embodiments 20 to 28, wherein the composition comprises particles, formed by the ternary intermetallic compound, having a particle size D10 in the range of from 35 to 59, preferably from 37 to 57 nm, more preferably from 39 to 55 nm, more preferably from 41 to 53 nm, more preferably from 43 to 51 nm, and more preferably from 45 to 49 nm.
34. The composition of any one of embodiments 20 to 28 or embodiment 33, wherein the composition comprises particles, formed by the ternary intermetallic compound, having a particle size D50 in the range of from 55 to 79 nm, preferably from 57 to 77 nm, more preferably from 59 to 75 nm, more preferably from 61 to 73 nm, more preferably from 63 to 71 nm, and more preferably from 65 to 69 nm.
35. The composition of any one of embodiments 20 to 28 or embodiment 33 or 34, wherein the composition comprises particles, formed by the ternary intermetallic compound, having a particle size D90 in the range of from 108 to 152 nm, preferably from 112 to 148 nm, more preferably from 116 to 144 nm, more preferably from 120 to 140 nm, more preferably from 124 to 136 nm, and more preferably from 128 to 132 nm.
36. The composition of any one of embodiments 20 to 28, wherein the composition comprises particles, formed by the ternary intermetallic compound, having a particle size D10 in the range of from 41 to 53 nm, preferably from 43 to 51 nm, and more preferably from 45 to 49 nm;
   wherein the particle size D50 is in the range of from 61 to 73 nm, preferably from 63 to 71 nm, and more preferably from 65 to 69 nm; and
   wherein the particle size D90 in the range of from 120 to 140 nm, preferably from 124 to 136 nm, and more preferably from 128 to 132 nm.
37. The composition of any one of embodiments 20 to 36, wherein in the composition, the crystallite size, determined via XRD using the Scherer equation as described in Reference Example 1.2 herein, is in the range of from 8 to 30 nm.
38. The composition of any one of embodiments 20 to 37, having a BET specific surface area, determined as described in Reference Example 1.3 herein, is in the range of from 150 to 400 m²/g.
39. The composition of any one of embodiments 1 to 38, wherein at least 99 weight-%, preferably at least 99.5 weight-%, more preferably at least 99.9 weight-% of the composition consist of the ternary intermetallic compound and the porous oxidic support material.
40. The composition any one of embodiments 1 to 39, wherein the intermetallic compound is a Heusler phase.
41. The composition of any one of embodiments 1 to 40, wherein the intermetallic compound is supported on the porous oxidic material in the form of particles.
42. A process for preparing an intermetallic compound supported on a porous oxidic support material according to any one of embodiments 1 to 41, comprising
   (i) preparing a liquid mixture comprising a source of X, a source of Y, a source of Z, and a source of the porous oxidic support material;
   (ii) removing the liquid phase from the mixture prepared in (i);
   (iii) heating the mixture obtained from (ii) in a reducing atmosphere, obtaining the intermetallic compound supported on a porous oxidic support material.
43. The process of embodiment 42, wherein the source of X is selected from the group consisting of salts of X, wherein the salts of X are preferably selected from the group consisting of acetates, acetylacetonates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof, more preferably from the group consisting of acetates, acetylacetonates, nitrates, chlorides, bromides, fluorides, and mixtures of two or more thereof, wherein more preferably, more preferably from the group consisting of acetates, acetylacetonates, nitrates and chlorides.
44. The process of embodiment 42 or 43, wherein the source of Y is selected from the group consisting of salts of Y, wherein the salts of Y are preferably selected from the group consisting of acetates, acetylacetonates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof, more preferably from the group consisting of acetates, acetylacetonates, nitrates, chlorides, bromides, fluorides, and mixtures of two or more thereof, more preferably from the group consisting of acetates, acetylacetonates, and nitrates.
45. The process of any one of embodiments 42 to 44, wherein the source of Z is selected from the group consisting of salts of Z, wherein the salts of Z are preferably selected from the group consisting of C1-C4 alkoxides, acetates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof, more preferably from the group consisting of C₂-C₃ alkoxides, acetates, nitrates, chlorides, bromides, fluorides, and mixtures of two or more thereof, more preferably from the group consisting of ethoxides, acetates, nitrates, and chlorides.
46. The process any one of embodiments 42 to 45, wherein source of the porous oxidic support material comprises one or more of silica, alumina, titania, zirconia, a mixed oxide of one or more Si, Al, Ti, and Zr, and a mixture of two or more thereof.
47. The process of embodiment 46, wherein at least 99 weight-%, preferably at least 99.5 weight-%, more preferably at least 99.9 weight-% of the porous oxidic support material consist of one or more of silica, alumina, titania, zirconia, a mixed oxide of one or more Si, Al, Ti, and Zr, and a mixture of two or more thereof.
48. The process of embodiment 47, wherein at least 99 weight-%, preferably at least 99.5 weight-%, more preferably at least 99.9 weight-% of the porous oxidic support material consist of the mixed oxide of Si and Al.
49. The process of embodiment 47 or 48, wherein the mixed oxide of Si and Al is a zeolitic material.
50. The process of embodiment 49, wherein the zeolitic material has a framework type which is ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEl, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SVV, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, a mixture of two or more of these framework types, or a mixed framework type thereof.
51. The process of embodiment 49 or 50, wherein the zeolitic material has framework type MFI.
52. The process of any one of embodiments 49 to 51, wherein the zeolitic material comprises, preferably is a zeolite ZSM-5.
53. The process of embodiment 47, wherein the porous oxidic support material comprises silica.
54. The process of embodiment 53, wherein at least 99 weight-%, preferably at least 99.5 weight-%, more preferably at least 99.9 weight-% of the porous oxidic support material consist of silica.
55. The process of embodiment 53 or 54, wherein the silica comprises, preferably is fumed silica.
56. The process of any one of embodiments 53 to 55,
   wherein the silica has a BET specific surface area, determined as described in Reference Example 1.3, in the range of from 300 to 500 m²/g, preferably in the range of from 350 to 450 m²/g; and/or
   wherein the silica has a total pore volume, determined as described in Reference Example 1.3, in the range of from 0.4 to 0.5 ml/g, preferably in the range of from 0.42 to 0.48 ml/g; and/or
   wherein the silica has an average pore size, determined as described in Reference Example 1.3, in the range of from 4 to 5 nm, preferably in the range of from 4.2 to 4.8 nm
57. The process of any one of embodiments 42 to 56, wherein (i) comprises
   (i.1) preparing a liquid mixture comprising a source of X, a source of Y, a source of Z, and a solvent;
   (i.2) admixing the source of the porous oxidic support material with the mixture prepared in (i.1).
58. The process of embodiment 57, wherein the solvent according to (i.1) is a polar solvent, preferably one or more polar protic solvents, more preferably selected from the group consisting of water, C₁ alcohols, C₂ alcohols, C₃ alcohols, C₄ alcohols, and mixtures of two or more thereof, more preferably selected from the group consisting of water, C₁ alcohols, C₂ alcohols, C₃ alcohols, and mixtures of two or more thereof, wherein more preferably, the solvent is one or more of water, methanol and ethanol, wherein more preferably, the solvent according to (ii) comprises, preferably is, methanol.
59. The process of embodiment 57 or 58, wherein (i.2) comprises preparing a liquid mixture comprising a solvent and the source of the porous oxidic support material and admixing the liquid mixture with the mixture prepared in (i.1).
60. The process of embodiment 59, wherein the solvent according to (i.2) is a polar solvent, preferably one or more polar protic solvents, more preferably selected from the group consisting of water, C₁ alcohols, C₂ alcohols, C₃ alcohols, C₄ alcohols, and mixtures of two or more thereof, more preferably selected from the group consisting of water, C₁ alcohols, C₂ alcohols, C₃ alcohols, and mixtures of two or more thereof, wherein more preferably, the solvent is one or more of water, methanol and ethanol, wherein more preferably, the solvent according to (i.2) comprises, preferably is, methanol.
61. The process of embodiment 59 or 60, wherein the solvent according to (i.2) is the solvent according to (i.1).
62. The process of any one of embodiments 42 to 61, wherein removing the liquid phase from the mixture according to (ii) comprises heating the mixture prepared in (i), preferably heating to a temperature of the mixture in the range of from 70 to 150° C., preferably in the range of from 80 to 140° C., more preferably from 90 to 130° C., more preferably from 100 to 120° C., preferably in the range of from 08 to 1.2 bar(abs), more preferably in the range of from 0.9 to 1.1 bar(abs).
63. The process of embodiment 62, wherein according to (ii) and prior to heating, the mixture prepared in (i) is subjected to evaporation, preferably at a pressure in the range of from 2 to 500 mbar(abs), preferably in the range of from 5 to 200 mbar(abs), more preferably in the range of from 10 to 100 mbar(abs).
64. The process of embodiment 63, wherein during evaporation, the temperature of the mixture is adjusted to a value in the range of from 20 to 60° C., preferably in the range of from 30 to 50° C.
65. The process of any one of embodiments 42 to 64, wherein the reducing atmosphere according to (iii) comprises hydrogen) preferably comprises hydrogen and an inert gas, preferably nitrogen.
66. The process of embodiment 65, wherein in the reducing atmosphere, the volume ratio of hydrogen relative to the inert gas is in the range of from 30:70 to 70:30, preferably in the range of from 40:60 to 60:40.
67. The process of any one of embodiments 42 to 66, wherein according to (iii), the mixture is heated to a temperature of the reducing atmosphere in the range of from 400 to 1,100° C., preferably in the range of from 500 to 1,000° C.
68. The process of any one of embodiments 42 to 67, wherein according to (iii), the mixture is heated to a temperature of the reducing atmosphere in the range of from 800 to 1,000° C., preferably in the range of from 850 to 1000° C.
69. The process of embodiment 67 or 68, wherein according to (iii), the mixture is heated at a temperature ramp in the range of from 0.1 to 15 K/min, preferably in the range of from 0.3 to 12 K/min.
70. The process of any one of embodiments 67 to 69, wherein according to (iii), the mixture is heated at a temperature ramp in the range of from 0.4 to 7 K/min, preferably in the range of from 0.5 to 5 K/min.
71. The process of any one of embodiments 67 to 69, wherein according to (iii), the mixture is heated at a temperature ramp in the range of from 8 to 12 K/min, preferably in the range of from 9 to 11 K/min.
72. The process of any one of embodiments 67 to 71, wherein according to (iii), the mixture is kept at the temperature for a period of time in the range of from 0.5 to 20 h, preferably in the range of from 1 to 15 h, more preferably in the range of from 2 to 10 h.
73. The process of any one of embodiment 42 to 72, further comprising
   (iv) cooling the intermetallic compound supported on a porous oxidic material, obtained from (iii).
74. A composition of any one of embodiments 1 to 41, obtainable or obtained or preparable or prepared by a process according to any one of embodiments 42 to 73.
75. Use of the composition according to any one of embodiments 1 to 41 or 74 as a catalytically active material, preferably for an oxidation reaction, a hydrogenation reaction, a dehydrogenation reaction, and/or a condensation reaction.
76. The use of embodiment 75, wherein the hydrogenation reaction comprises the hydrogenation of an aldehyde, preferably cinnamaldehyde.
77. The use of embodiment 75 or 76, wherein the dehydrogenation reaction comprises the dehydrogenation of an alkane, preferably propane.
78. The use of any one of embodiments 75 to 77, wherein the oxidation reaction comprises the oxidation of an alkane, preferably a cyclic alkane, more preferably cyclohexane.
79. The use of any one of embodiments 75 to 77, wherein the condensation reaction comprises the condensation of a carbonyl compound with a methylene group containing compound, wherein the condensation reaction is preferably a Knoevenagel condensation reaction.
80. A method for catalytically converting an organic compound, comprising bringing the organic compound in contact with a catalyst which comprises a composition according to any one of embodiments 1 to 41 or 74 as a catalytically active material, wherein the converting of the organic compound comprises an oxidation reaction, a hydrogenation reaction, a dehydrogenation reaction, and/or a condensation reaction.
81. The method of embodiment 80, wherein the hydrogenation reaction comprises the hydrogenation of an aldehyde, preferably cinnamaldehyde.
82. The method of embodiment 80 or 81, wherein the dehydrogenation reaction comprises the dehydrogenation of an alkane, preferably propane.
83. The method of any one of embodiments 80 to 82, wherein the oxidation reaction comprises the oxidation of an alkane, preferably a cyclic alkane, more preferably cyclohexane.
84. The method of any one of embodiments 80 to 83, wherein the condensation reaction comprises the condensation of a carbonyl compound with a methylene group containing compound, wherein the condensation reaction is preferably a Knoevenagel condensation reaction.
85. A catalyst, comprising a composition according to any one of embodiments 1 to 41 or 74 as a catalytically active material, and optionally one or more further catalytically active materials and/or one or more matrix materials.

The present invention is further illustrated by the following examples, comparative examples, and reference examples.

### EXAMPLES

### Reference Example 1.1: Determination of the Particle Size Via TEM

Samples for Transmission Electron Microscopy (TEM) were prepared on ultra-thin carbon TEM carriers. The powder was therefore dispersed in ethanol. One drop of the dispersion was applied between two glass objective slides and gently dispersed. The TEM carrier film was subsequently dipped on the resulting thin film. The samples were imaged by TEM using a Tecnai Osiris machine (FEI Company, Hillsboro, USA) operated at 200 keV under bright-field as well as high-angle annular dark-field scanning TEM (HAADF-STEM) conditions. Chemical composition maps were acquired by energy-dispersive x-ray spectroscopy (EDXS). Images and elemental maps were evaluated using the iTEM (Olympus, Tokyo, Japan, version: 5.2.3554) as well as the Esprit (Bruker, Billerica, USA, version 1.9) software packages. Particle size distributions were evaluated using the ParticleSizer plugin for FIJI.

### Reference Example 1.2: X-Ray Powder Diffraction and Determination of the Crystallite Size

The X-ray powder diffraction (XRD) measurements were carried out with a D 5005 type diffractometer of Siemens/Bruker AXS using a Cu Kalpha Source (lambda=0.15405 nm). The source was operated at 35 kV and 25 mA and the data were collected in a 2theta range from 3 to 110° with a step size of 0.1° (2theta). The crystallite sizes were determined by the Scherer equation using peaks at 79° 2theta. A Gaussian fit was used to determine the full width at half maximum (FWHM).

### Reference Example 1.3: Determination of the BET Specific Surface Area, the Total Pore Volume and the Average Pore Size

The BET specific surface area was determined according to DIN 66131 via nitrogen adsorption/desorption at a temperature of 77 K. The total pore volume was determined via mercury intrusion porosimetry according to DIN 66133. The average pore size was determined via mercury intrusion porosimetry according to DIN 66133.

### Reference Example 1.4: Starting Materials

1.4.1 Metal Sources

The following materials were employed for preparing the intermetallic compounds (see Table 1 below):

TABLE 1
Starting materials

| | | molecular |
|---|---|---|
| | | weight/ |
| precursor | formula | (g/mol) |
| chromium(III) nitrate nonahydrate | Cr(NO₃)₃•9 H₂O | 400.15 |
| cobalt(II) nitrate hexahydrate | Co(NO₃)₂•6 H₂O | 291.04 |
| gallium(III) nitrate 5.5hydrate | Ga(NO₃)₃•5.5 H₂O | 354.82 |
| aluminum nitrate nonahydrate | Al(NO₃)₃•9 H₂O | 375.13 |
| zinc nitrate tetrahydrate | Zn(NO₃)₂•4 H₂O | 261.45 |
| indium(III) nitrate dihydrate | In(NO₃)₃•2 H₂O | 336.86 |
| copper(II) nitrate pentahemihydrate | Cu(NO₃)₂•2.5 H₂O | 232.59 |
| antimony(III) acetate | (CH₃CO₂)₃Sb | 298.89 |
| tin(II) chloride dihydrate | SnCl₂•2 H₂O | 225.64 |
| nickel(II) nitrate hexahydrate | Ni(NO₃)₂•6 H₂O | 290.80 |

1.4.2 Porous Oxidic Materials
a) Silica: As porous oxidic support material, fumed silica according to the following specification was employed: Sigma Aldrich, lot no. S5130 (particle size=0.007 micrometer, BET specific surface area=370-420 m²/g, density=2.3 lb/ft³ (1 lb/ft²=16.018463 kg/m³)).
b) Zeolite: As a further oxidic support material, a zeolite ZSM-5 (framework type MFI) was employed. The zeolitic material was prepared as follows: a solution consisting of 12.72 g sulfuric acid and 1.8 g sodium aluminate in 240 g water was produced. Under stirring 160 g sodium silicate solution was slowly added. Once a homogenous synthesis gel has formed a solution of 19.2 g tetrapropylammonium bromide and 32 g water were slowly added. The synthesis gel was stirred at room temperature for 0.5 h. Afterwards the crystallization was carried out in a rotating stainless steel autoclave (50 rpm) with a Teflon inlay at 180° C. for 72 h. The BET specific surface area was 452 m²/g. The X-Ray diffraction pattern of the ZSM-5 zeolitic material is shown in FIG. 39 for 2theta in the range of from 3 to 50°. An SEM image is shown in FIG. 40. The respectively prepared zeolite ZSM-5 had a molar ratio of SiO₂:Al₂O₃=61.

### Example 1: Preparation of Ternary Intermetallic Compounds Supported on a Porous Oxidic Support

According to Example 1, the following ternary intermetallic compounds supported on a porous oxidic support were prepared (see Table 2 below). In Example 1.1, the typical process is disclosed.

TABLE 2
Compositions prepared according to Example 1

| | | | | metal | mass | | mass X | | mass Y | | mass Z |
|---|---|---|---|---|---|---|---|---|---|---|---|
| composition | | | | content/ | support/ | amount | precursor/ | amount | precursor/ | amount | precursor/ |
| X | Y | Z | support | wt.-% | g | X/mmol | g | Y/mmol | g | Z/mmol | g |
| Co | Cr | Al | ZSM-5 | 5 | 2.15 | 1.15 | 0.33 | 0.57 | 0.23 | 0.58 | 0.22 |
| Co | Cr | Ga | ZSM-5 | 5 | 2.22 | 0.98 | 0.28 | 0.49 | 0.20 | 0.49 | 0.17 |
| Co | Cr | In | ZSM-5 | 5 | 2.19 | 0.81 | 0.24 | 0.41 | 0.16 | 0.41 | 0.14 |
| Co | Cr | Zn | ZSM-5 | 5 | 2.17 | 0.97 | 0.28 | 0.49 | 0.19 | 0.49 | 0.13 |
| Cu | Co | Al | ZSM-5 | 5 | 2.33 | 1.15 | 0.27 | 0.58 | 0.17 | 0.58 | 0.22 |
| Cu | Co | Ga | ZSM-5 | 5 | 2.11 | 0.87 | 0.20 | 0.43 | 0.13 | 0.43 | 0.15 |
| Cu | Co | In | ZSM-5 | 5 | 2.10 | 0.47 | 0.11 | 0.47 | 0.14 | 0.47 | 0.16 |
| Cu | Co | Zn | ZSM-5 | 5 | 2.23 | 0.94 | 0.22 | 0.47 | 0.14 | 0.47 | 0.12 |

### Example 1.1: Preparation of 5 Weight-% Co₂CrAl Supported on Zeolite ZSM-5

Co(NO₃)₂.6H₂O (1.57 g, 5.39 mmol), Cr(NO₃)₃.9H₂O (1.08 g, 2.69 mmol) and Al(NO₃)₃.9H₂O (1.01 g, 2.69 mmol) were dissolved in 80 ml methanol. A round bottom flask containing the solution was placed in an ultrasonic bath and treated for 30 minutes. ZSM-5 (2.15 g) and 100 ml methanol were supplied to another round bottom flask and sonicated for 30 min. The precursor solution was added to the fumed silica suspension and sonicated for 30 min at room temperature. Then, the methanol was removed in a rotary evaporator with the water bath temperature adjusted to 40° C. The residue was dried at 110° C. for 18 h. The solid was grounded to a powder and filled into a vertically arranged flow-type quartz reactor. The reactor was thoroughly purged with nitrogen (100 ml/min) for 10 minutes at room temperature. The powder was then reduced in a mixture of flowing hydrogen and nitrogen (100 ml/min hydrogen and 100 ml/min nitrogen) at 900° C. The maximum temperature was achieved by heating rate of 10 K/min⁻¹ and kept constant for 8 h. Finally, the samples were cooled to room temperature. The crystal structure of the prepared ternary intermetallic compounds was investigated by X-ray powder diffraction. The X-ray diffraction pattern of 5 weight-% Co₂CrAl/ZSM-5 for the range 2theta=3-100° is shown in FIG. 1. The sharp reflections between 2theta=40-100° are caused by crystalline nanoparticles. The diffraction peaks between 2theta=3-40° can be assigned to the ZSM-5 support.

### Example 1.2: Preparation of 5 Weight-% Co₂CrGa Supported on Zeolite ZSM-5

The preparation was carried out analogously to Example 1.1.

The crystal structure of the intermetallic compounds was investigated by X-ray powder diffraction. The X-ray diffraction pattern of 5 weight-% Co₂CrGa/ZSM-5 for the range 2theta=3-100° is shown in FIG. 2. The sharp reflections between 2theta=40-100° are caused by crystalline nanoparticles. The diffraction peaks between 2theta=3-40° can be assigned to the ZSM-5 support.

### Example 1.3: Preparation of 5 Weight-% Co₂CrIn Supported on Zeolite ZSM-5

The preparation was carried out analogously to Example 1.1. The crystal structure of the intermetallic compounds was investigated by X-ray powder diffraction. The X-ray diffraction pattern of 5 weight-% Co₂CrIn/ZSM-5 for the range 2theta=3-100° is shown in FIG. 3. The sharp reflections between 2theta=40-100° are caused by crystalline nanoparticles. The diffraction peaks between 2theta=3-40° can be assigned to the ZSM-5 support.

### Example 1.4: Preparation of 5 Weight-% Co₂CrZn Supported on Zeolite ZSM-5

The preparation was carried out analogously to Example 1.1. The crystal structure of the intermetallic compounds was investigated by X-ray powder diffraction. The X-ray diffraction pattern of 5 weight-% Co₂CrZn/ZSM-5 for the range 2theta=3-100° is shown in FIG. 4. The sharp reflections between 2theta=40-100° are caused by crystalline nanoparticles. The diffraction peaks between 2theta=3-40° can be assigned to the ZSM-5 support.

### Example 1.5: Preparation of 5 Weight-% Cu₂CoAl Supported on Zeolite ZSM-5

The preparation was carried out analogously to Example 1.1. The crystal structure of the intermetallic compounds was investigated by X-ray powder diffraction. The X-ray diffraction pattern of 5 weight-% Cu₂CoAl/ZSM-5 for the range 2theta=3-100° is shown in FIG. 5. The sharp reflections between 2theta=40-100° are caused by crystalline nanoparticles. The diffraction peaks between 2theta=3-40° can be assigned to the ZSM-5 support.

### Example 1.6: Preparation of 5 Weight-% Cu₂CoGa Supported on Zeolite ZSM-5

The preparation was carried out analogously to Example 1.1. The crystal structure of the intermetallic compounds was investigated by X-ray powder diffraction. The X-ray diffraction pattern of 5 wt.-% Cu₂CoGa/ZSM-5 for the range 2theta=3-100° is shown in FIG. 6. The sharp reflections between 2theta=40-100° are caused by crystalline nanoparticles. The diffraction peaks between 2theta=3-40° can be assigned to the ZSM-5 support.

### Example 1.7: Preparation of 5 Weight-% Cu₂CoIn Supported on Zeolite ZSM-5

The preparation was carried out analogously to Example 1.1. The crystal structure of the intermetallic compounds was investigated by X-ray powder diffraction. The X-ray diffraction pattern of 5 wt.-% Cu₂CoIn/ZSM-5 for the range 2theta=3-100° is shown in FIG. 7. The sharp reflections between 2theta=40-100° are caused by crystalline nanoparticles. The diffraction peaks between 2theta=3-40° can be assigned to the ZSM-5 support.

### Example 1.8: Preparation of 5 Weight-% Cu₂CoZn Supported on Zeolite ZSM-5

The preparation was carried out analogously to Example 1.1. The crystal structure of the intermetallic compounds was investigated by X-ray powder diffraction. The X-ray diffraction pattern of 5 weight-% Cu₂CoZn/ZSM-5 for the range 2theta=3-100° is shown in FIG. 8. The sharp reflections between 2theta=40-100° are caused by crystalline nanoparticles. The diffraction peaks between 2theta 3-40° can be assigned to the ZSM-5 support.

### Example 2: Preparation of Ternary Intermetallic Compounds Supported on a Porous Oxidic Support

### Example 2.1: Preparation of 30 Weight-% Cu₂NiZ Supported on SiO₂

All combinations prepared by the procedure as described in Example 2.1.1 below were prepared with a total metal content of 30 weight-% and a metal stoichiometry of X:Y:Z=2:1:1.

### Example 2.1.1: Preparation of 30 Weight-% Cu₂NiAl Supported on SiO₂

Cu(NO₃)₂.2.5H₂O (1.96 g, 8.44 mmol), Ni(NO₃)₂.6H₂O (1.23 g, 4.22 mmol) and SnCl₂.2H₂O (0.95 g, 4.22 mmol) were dissolved in 80 ml methanol. A round bottom flask containing the solution was placed in an ultrasonic bath and treated for 30 min. Fumed silica (3.00 g, 7 nm) and 10 ml methanol were supplied to another round bottom flask and sonicated for 30 min. The precursor solution was added to the fumed silica suspension and sonicated for 30 min at room temperature. Then, the methanol was removed in a rotary evaporator with the water bath temperature adjusted to 40° C. The residue was dried at 110° C. for 18 h. The solid was grounded to a powder and filled into a vertically arranged flow-type quartz reactor. The reactor was thoroughly purged with nitrogen (100 ml/min) for 10 min at room temperature. The powder was then reduced in a mixture of flowing hydrogen and nitrogen (100 ml/min hydrogen and 100 ml/min nitrogen) at 880° C. The detailed temperature program for the reducing method is given in Table 3 below. Finally, the samples were cooled to room temperature.

TABLE 3
Temperature program for the reduction according to Example 2.1.1

| temperature | | |
|---|---|---|
| ramp/(K/min) | Temperature/° C. | Holding temperature for . . . min |
| — | 25 | — |
| 5 | 100 | — |
| 0.5 | 210 | 60 |
| 5 | 880 | 180 |

The crystal structure of the intermetallic compounds was examined by X-ray powder diffraction. The X-ray diffraction pattern for 30 weight-% Cu₂NiAl/SiO₂ for the angle range 2theta=15-100° is shown in FIG. 9. The sharp reflections between 2theta=20-100° are caused by crystalline nanoparticles. The wide diffraction peak between 2theta=20-35° can be assigned to the support fumed silica.

### Example 2.1.2: Preparation of 30 Weight-% Cu₂NiGa Supported on SiO₂

The preparation was carried out analogously to Example 2.1.1. The crystal structure of the intermetallic compounds was examined by X-ray powder diffraction. The X-ray diffraction pattern for 30 weight-% Cu₂NiGa/SiO₂ for the angle range 2theta=15-100° is shown in FIG. 10. The sharp reflections between 2theta=20-100° are caused by crystalline nanoparticles. The wide diffraction peak between 2theta=20-35° can be assigned to the support fumed silica.

### Example 2.1.3: Preparation of 30 Weight-% Cu₂NiIn Supported on SiO₂

The preparation was carried out analogously to Example 2.1.1. The crystal structure of the intermetallic compounds was examined by X-ray powder diffraction. The X-ray diffraction pattern for 30 weight-% Cu₂NiIn/SiO₂ for the angle range 2theta=15-100° is shown in FIG. 11. The sharp reflections between 2theta=20-100° are caused by crystalline nanoparticles. The wide diffraction peak between 2theta=20-35° can be assigned to the support fumed silica.

### Example 2.1.4: Preparation of 30 Weight-% Cu₂NiSb Supported on SiO₂

The preparation was carried out analogously to Example 2.1.1. The crystal structure of the intermetallic compounds was examined by X-ray powder diffraction. The X-ray diffraction pattern for 30 weight-% Cu₂NiSbISiO₂ for the angle range 2theta=15-100° is shown in FIG. 12. The sharp reflections between 2theta=20-100° are caused by crystalline nanoparticles. The wide diffraction peak between 2theta=20-35° can be assigned to the support fumed silica.

### Example 2.1.5: Preparation of 30 Weight-% Cu₂NiSi Supported on SiO₂

The preparation was carried out analogously to Example 2.1.1. The crystal structure of the intermetallic compounds was examined by X-ray powder diffraction. The X-ray diffraction pattern of 30 weight-% Cu₂NiSi/SiO₂ for the range 2theta=15-100° is shown in FIG. 13. The sharp reflections between 2theta=20-100° are caused by crystalline nanoparticles. The wide diffraction peak between 2theta=20-35° can be assigned to the support fumed silica.

### Example 2.1.6: Preparation of 30 Weight-% Cu₂NiSn Supported on SiO₂

The preparation was carried out analogously to Example 2.1.1. The crystal structure of the intermetallic compounds was examined by X-ray powder diffraction. The X-ray diffraction pattern of 30 weight-% Cu₂NiSn/SiO₂ for the range 2theta=15-100° is shown in FIG. 14. The sharp reflections between 2theta=20-100° are caused by crystalline nanoparticles. The wide diffraction peak between 2theta=20-35° can be assigned to the support fumed silica. TEM images (see FIG. 29) of show particles in the range from a few nanometer up to 400 nm. The particles containing Cu/Ni/Sn are of spherical shape.

### Example 2.2: Preparation of 30 Weight-% Cu₂NiSn Supported on SiO₂ at Different Reduction Temperatures

All combinations prepared by the procedure as described in Example 2.2 were prepared with a total metal content of 30 weight-% and a metal stoichiometry of X:Y:Z=2:1:1 and varying reduction temperatures.

Cu(NO₃)₂.2.5H₂O (1.96 g, 8.44 mmol) Ni(NO₃)₂.6H₂O (1.23 g, 4.22 mmol) and SnCl₂.2H₂O (0.95 g, 4.22 mmol) were dissolved in 80 ml methanol. A round bottom flask containing the solution was placed in an ultrasonic bath and treated for 30 min. Fumed silica (3.00 g, 7 nm) and 10 ml methanol were supplied to another round bottom flask and sonicated for 30 min. The precursor solution was added to the fumed silica suspension and sonicated for 30 min at room temperature. Then, the methanol was removed in a rotary evaporator with the water bath temperature adjusted to 40° C. The residue was dried at room temperature for 18 h. The solid was grounded to a powder and filled into a vertically arranged flow-type quartz reactor. The reactor was thoroughly purged with nitrogen (100 ml/min) for 10 min at room temperature. The powder was then reduced in a mixture of flowing hydrogen and nitrogen (100 ml/min hydrogen and 100 ml/min nitrogen) at the respective temperature indicated in the Examples 2.2.1 to 2.2.6. The maximum temperature was achieved using a heating rate of 10 K/min and kept constant for 3 h. Finally, the samples were cooled to room temperature.

### Example 2.2.1: Preparation of 30 Weight-% Cu₂NiSn Supported on SiO₂ at a Maximum Temperature of 500° C.

The crystal structure of the Heusler compounds was examined by X-ray powder diffraction. The X-ray diffraction pattern for 30 weight-% Cu₂NiSn/SiO₂ for the angle range 2theta=15-100° is shown in FIG. 15. The sharp reflections between 2theta=20-100° are caused by crystalline nanoparticles. The broad diffraction peak between 2theta=20-35° can be assigned to the support fumed silica. The characteristic signals for the Heusler L2₁ phase are observed at 25.9°, 30°, 42.9°, 62.4° and 78.9°.

### Example 2.2.2: Preparation of 30 Weight-% Cu₂NiSn Supported on SiO₂ at a Maximum Temperature of 600° C.

The crystal structure of the Heusler compounds was examined by X-ray powder diffraction. The X-ray diffraction pattern for 30 weight-% Cu₂NiSn/SiO₂ for the angle range 2theta=15-100° is shown in FIG. 16. The sharp reflections between 2theta=20-100° are caused by crystalline nanoparticles. The broad diffraction peak between 2theta=20-35° can be assigned to the support fumed silica. The characteristic signals for the Heusler L2₁ phase are observed at 25.9°, 30°, 42.9°, 62.4° and 78.9°.

### Example 2.2.3: Preparation of 30 Weight-% Cu₂NiSn Supported on SiO₂ at a Maximum Temperature of 700° C.

The crystal structure of the Heusler compounds was examined by X-ray powder diffraction. The X-ray diffraction pattern for 30 weight-% Cu₂NiSn/SiO₂ for the angle range 2theta=15-100° is shown in FIG. 17. The sharp reflections between 2theta=20-100° are caused by crystalline nanoparticles. The broad diffraction peak between 2theta=20-35° can be assigned to the support fumed silica. The characteristic signals for the Heusler L2₁ phase are observed at 25.9°, 30°, 42.9°, 62.4° and 78.9°.

### Example 2.2.4: Preparation of 30 Weight-% Cu₂NiSn Supported on SiO₂ at a Maximum Temperature of 800° C.

The crystal structure of the Heusler compounds was examined by X-ray powder diffraction. The X-ray diffraction pattern for 30 weight-% Cu₂NiSn/SiO₂ for the angle range 2theta=15-100° is shown in FIG. 18. The sharp reflections between 2theta=20-100° are caused by crystalline nanoparticles. The broad diffraction peak between 2theta=20-35° can be assigned to the support fumed silica. The characteristic signals for the Heusler L2₁ phase are observed at 25.9°, 30°, 42.9°, 62.4° and 78.9°.

### Example 2.2.5: Preparation of 30 Weight-% Cu₂NiSn Supported on SiO₂ at a Maximum Temperature of 900° C.

The crystal structure of the Heusler compounds was examined by X-ray powder diffraction. The X-ray diffraction pattern for 30 weight-% Cu₂NiSn/SiO₂ for the angle range 2theta=15-100° is shown in FIG. 19. The sharp reflections between 2theta=20-100° are caused by crystalline nanoparticles. The broad diffraction peak between 2theta=20-35° can be assigned to the support fumed silica. The characteristic signals for the Heusler L2₁ phase are observed at 25.9°, 30°, 42.9°, 62.4° and 78.9°. It is observed that heating above 800° C. avoids phase impurities.

### Example 2.2.6: Preparation of 30 Weight-% Cu₂NiSn Supported on SiO₂ at a Maximum Temperature of 1000° C.

The crystal structure of the Heusler compounds was examined by X-ray powder diffraction. The X-ray diffraction pattern for 30 weight-% Cu₂NiSn/SiO₂ for the angle range 2theta=15-100° is shown in FIG. 20. The sharp reflections between 2theta=20-100° are caused by crystalline nanoparticles. The broad diffraction peak between 2theta=20-35° can be assigned to the support fumed silica. The characteristic signals for the Heusler L2₁ phase are observed at 25.9°, 30°, 42.9°, 62.4° and 78.9°. It is observed that heating above 800° C. avoids phase impurities.

### Example 2.3: Preparation of Cu₂NiSn Supported on SiO₂ with Different Metal Content

All combinations prepared by the procedure as described in Example 2.3 were prepared with a metal stoichiometry of X:Y:Z=2:1:1 and varying metal content, as shown in Table 4 below:

Table 4
Compositions prepared according to Example 2.3

| | | | | metal | | | mass X | | mass Y | | mass Z |
|---|---|---|---|---|---|---|---|---|---|---|---|
| composition | | | | content/ | Example | amount | precursor/ | amount | precursor/ | amount | precursor/ |
| X | Y | Z | support | wt.-% | # | X/mmol | g | Y/mmol | g | Z/mmol | g |
| Cu | Ni | Sn | SiO₂ | 30 | 2.3.1 | 8.44 | 1.96 | 4.22 | 1.23 | 4.22 | 0.95 |
| Cu | Ni | Sn | SiO₂ | 20 | 2.3.2 | 4.93 | 1.15 | 2.46 | 0.72 | 2.46 | 0.56 |
| Cu | Ni | Sn | SiO₂ | 15 | 2.3.3 | 3.48 | 0.81 | 1.74 | 0.51 | 1.74 | 0.39 |
| Cu | Ni | Sn | SiO₂ | 10 | 2.3.4 | 2.19 | 0.51 | 1.09 | 0.32 | 1.09 | 0.25 |
| Cu | Ni | Sn | SiO₂ | 5 | 2.3.5 | 1.04 | 0.24 | 0.52 | 0.15 | 0.52 | 0.12 |
| Cu | Ni | Sn | SiO₂ | 2 | 2.3.6 | 0.40 | 0.09 | 0.20 | 0.06 | 0.20 | 0.05 |
| Cu | Ni | Sn | SiO₂ | 1 | 2.3.7 | 0.20 | 0.05 | 0.10 | 0.03 | 0.10 | 0.02 |

The compositions were prepared as follows: Cu(NO₃)₂.2.5H₂O (respective amount according to Table 4) Ni(NO₃)₂.6H₂O (respective amount according to Table 4) and SnCl₂.2H₂O (respective amount according to Table 4) were dissolved in 80 ml methanol. A round bottom flask containing the solution was placed in an ultrasonic bath and treated for 30 min. Fumed silica (3.00 g, 7 nm) and 10 ml methanol were supplied to another round bottom flask and sonicated for 30 min. The precursor solution was added to the fumed silica suspension and sonicated for 30 min at room temperature. Then, the methanol was removed in a rotary evaporator with the water bath temperature adjusted to 40° C. The residue was dried at room temperature for 18 h. The solid was grounded to a powder and filled into a vertically arranged flow-type quartz reactor. The reactor was thoroughly purged with nitrogen (100 ml/min) for 10 min at room temperature. The powder was then reduced in a mixture of flowing hydrogen and nitrogen (100 ml/min hydrogen and 100 ml/min nitrogen) at 800° C. The maximum temperature was achieved at a heating rate of 10 K/min and kept constant for 3 h. Finally, the samples were cooled to room temperature. The L2₁ structure cannot be undoubtedly verified for metal content below 10 wt.-%. The signal broadening is assumed to be resulting from decreasing the crystallite sizes. In FIG. 28, the crystallite size is shown as a function of the metal content of the Heusler compounds of Examples 2.3.1 to 2.3.4.

### Example 2.3.1: Preparation of Cu₂NiSn Supported on SiO₂ with a Metal Content of 30 Weight-%

The crystal structure of the Heusler compound was examined by X-ray powder diffraction. The X-ray diffraction pattern for 30 weight-% Cu₂NiSn/SiO₂ for the angle range 2theta=15-100° is shown in FIG. 21. The sharp reflections between 2theta=20-100° are caused by crystalline nanoparticles. The wide diffraction peak between 2theta=20-35° can be assigned to the support fumed silica. The characteristic signals for the L2₁ phase are observed at 25.9°, 30° 42.9°, 62.4° and 78.9°. In contrast to Example 2.1.6, TEM (see FIG. 30) show larger particles with a size of up to 2 micrometer. The two-phased janus particles contain Cu/Ni/Sn.

The particle size distribution of the Heusler compound, determined according to reference example 1.1, afforded a D10 value of about 47 nm, a D50 value of about 68 nm, and a D90 value of about 129 nm (see FIG. 41).

### Example 2.3.2: Preparation of Cu₂NiSn Supported on SiO₂ with a Metal Content of 20 Weight-%

The crystal structure of the Heusler compound was examined by X-ray powder diffraction. The X-ray diffraction pattern for 20 weight-% Cu₂NiSn/SiO₂ for the angle range 2theta=15-100° is shown in FIG. 22. The sharp reflections between 2theta=20-100° are caused by crystalline nanoparticles. The wide diffraction peak between 2theta=20-35° can be assigned to the support fumed silica. The characteristic signals for the L2₁ phase are observed at 25.9°, 30° 42.9°, 62.4° and 78.9°.

### Example 2.3.3: Preparation of Cu₂NiSn Supported on SiO₂ with a Metal Content of 15 Weight-%

The crystal structure of the Heusler compound was examined by X-ray powder diffraction. The X-ray diffraction pattern for 15 weight-% Cu₂NiSn/SiO₂ for the angle range 2theta=15-100° is shown in FIG. 23. The sharp reflections between 2theta=20-100° are caused by crystalline nanoparticles. The wide diffraction peak between 2theta=20-35° can be assigned to the support fumed silica. The characteristic signals for the L2₁ phase are observed at 25.9°, 30°, 42.9°, 62.4° and 78.9°.

### Example 2.3.4: Preparation of Cu₂NiSn Supported on SiO₂ with a Metal Content of 10 Weight-&

The crystal structure of the Heusler compound was examined by X-ray powder diffraction. The X-ray diffraction pattern for 10 weight-% Cu₂NiSn/SiO₂ for the angle range 2theta=15-100° is shown in FIG. 24. The sharp reflections between 2theta=20-100° are caused by crystalline nanoparticles. The wide diffraction peak between 2theta=20-35° can be assigned to the support fumed silica. The characteristic signals for the L2₁ phase are observed at 25.9°, 30°, 42.9°, 62.4° and 78.9°. TEM (see FIG. 31) show nano-particles in a range from 2 to 50 nm. The particles are homogeneously distributed on the support. Like in Example 2.3.1, some of the particles show janus shape.

The particle size distribution of the Heusler compound, determined according to reference example 1.1, afforded a D10 value of about 5 nm, a D50 value of about 7 nm, and a D90 value of about 13 nm (see FIG. 41).

### Example 2.3.5: Preparation of Cu₂NiSn Supported on SiO₂ with a Metal Content of 5 Weight-%

The crystal structure of the intermetallic compound was examined by X-ray powder diffraction. The X-ray diffraction pattern for 5 weight-% Cu₂NiSn/SiO₂ for the angle range 2theta=15-100° is shown in FIG. 25. The sharp reflections between 2theta=20-100° are caused by crystalline nanoparticles. The wide diffraction peak between 2theta=20-35° can be assigned to the support fumed silica.

### Example 2.3.6: Preparation of Cu₂NiSn Supported on SiO₂ with a Metal Content of 2 Weight-%

The crystal structure of the intermetallic compound was examined by X-ray powder diffraction. The X-ray diffraction pattern for 2 weight-% Cu₂NiSn/SiO₂ for the angle range 2theta=15-100° is shown in FIG. 26. The sharp reflections between 2theta=20-100° are caused by crystalline nanoparticles. The wide diffraction peak between 2theta=20-35° can be assigned to the support fumed silica.

### Example 2.3.7: Preparation of Cu₂NiSn Supported on SiO₂ with a Metal Content of 1 Weight-%

The crystal structure of the intermetallic compound was examined by X-ray powder diffraction. The X-ray diffraction pattern for 1 weight-% Cu₂NiSn/SiO₂ for the angle range 2theta=15-100° is shown in FIG. 27. The sharp reflections between 2theta=20-100° are caused by crystalline nanoparticles. The wide diffraction peak between 2theta=20-35° can be assigned to the support fumed silica.

### Example 3: Testing of the Supported Ternary Intermetallic Compounds of Example 1 as Catalytically Active Materials—Oxidation of Cyclohexane

The catalytic activities of the intermetallic compounds supported on ZSM-5 prepared according to Examples 1.1 to 1.4 (Co₂CrAl/ZSM-5, Co₂CrGa/ZSM-5, Co₂CrIn/ZSM-5, Co₂CrZn/ZSM-5) and Examples 1.5 to 1.8 (Cu₂CoAl/ZSM-5, Cu₂CoGa/ZSM-5, Cu₂CoIn/ZSM-5, Cu₂CoZn/ZSM-5) were tested in the oxidation of cyclohexane with molecular oxygen.

The oxidation of cyclohexane was carried out in a stainless steel autoclave with Teflon inlay, which was filled with 25 ml cyclohexane, 50 ml acetone as solvent and 250 mg of the supported intermetallic compound. Then the autoclave was pressurized with 2 MPa of synthetic air (20.5% O₂ in N₂) and heated up. After the reaction temperature of 150° C. was reached the reaction was carried out under stirring for 6 h. Product samples were analyzed in a gas chromatograph (HP 6890 Series) with integrated mass selective detector (HP 5973). For this purpose, 10 microL of the samples were mixed with 2 microL toluene (external standard) and diluted in 1000 microL acetone. The conditions for the gas chromatographic analysis of the products from the cyclohexane oxidation were chosen as follows:

sample volume: 4 microL

injector temperature: 200° C.

heating rate: start at 35° C., 10 min isothermal, heating rate 30 K/min to 200° C.

eluent: He

flow rate: 139.3 ml/min

column head pressure: 1.2 bar (abs)

split ratio: 50:1

column: CP-SIL 5 CB, 100% dimethylpolysiloxane
length=25 m, film thickness=0.25 micrometer

detector: MS

As products, cyclohexanone (CHO) and cyclohexanol (CHOL) were identified.

The yields of cyclohexanone and cyclohexanole obtained when using the intermetallic compounds of Examples 1.1 to 1.4 (Co₂CrAl/ZSM-5, Co₂CrGa/ZSM-5, Co₂CrIn/ZSM-5, Co₂CrZn/ZSM-5) are shown in FIG. 32, plotted versus the reaction time. In the reference reaction over ZSM-5, only a low yield was observed. For the ZSM-5-supported intermetallic compounds an increase in activity in comparison to the ZSM-5-type zeolite could be detected. The highest yield was found for Co₂CrAl/ZSM-5.

The yields of cyclohexanone and cyclohexanole obtained when using the intermetallic compounds of Examples 1.5 to 1.8 (Cu₂CoAl/ZSM-5, Cu₂CoGa/ZSM-5, Cu₂CoIn/ZSM-5, Cu₂CoZn/ZSM-5) are shown in FIG. 33, plotted versus the reaction time. In the reference reaction over zeolite ZSM-5, only a low yield was observed. Except for the Cu₂CoIn/ZSM-5 material the ZSM-5 supported intermetallic compounds showed an increase in activity in comparison to ZSM-5. The highest yield for both products was found for Cu₂CoZn/ZSM-5.

An overview of the results of the cyclohexane oxidation is shown in FIG. 34.

### Example 4: Testing of the Supported Ternary Intermetallic Compounds of Example 2 as Catalytically Active Materials

### Example 4.1 Hydrogenation of Cinnamaldehyde ((2E)-3-Phenylprop-2-Enal)

The catalytic activities of the synthesized supported intermetallic compounds were tested in the cinnamaldehyde hydrogenation reaction. In particular, the compounds of Examples 2.1.6 (30 weight-% Cu₂NiSn/SiO₂), Example 2.3.1 (30 weight-% Cu₂NiSn/SiO₂) and Example 2.3.4 (10 weight-% Cu₂NiSn/SiO₂) were tested.

A batch autoclave with magnetic stirring was supplied with 150 ml cyclohexane, 5 ml cinnamaldehyde, 1 ml tetradecane as internal standard and 0.5 g of the respective supported intermetallic compound. The autoclave was sealed and afterwards flushed 3 with 7 bar of nitrogen. After 3 times flushing with 20 bar(abs) hydrogen, the reactor was heated up to 150° C. By reaching 150° C., hydrogen was used to set the pressure to 50 bar(abs), which was defined as the start time of the reaction. At regular time intervals, the reaction mixture was analyzed by gas chromatography. The conditions for the gas chromatographic analysis of the products from the hydrogenation of cinnamaldehyde were chosen as follows:

stationary phase: CP-SIL 5 CB

length: 25 m

inner diameter: 250 micrometer

film thickness: 0.25 micrometer

oven temperature: start: 120° C. 1 min
7 K/min 160° C.
50 K/min 220° C. 3 min

inlet temperature: 220° C.

split ratio: 30:1

total flow rate: 32.6 ml/min

eluent: N₂

velocity: 30 cm/s

detector: flame ionization detector

makeup flow: 45 ml/min

hydrogen flow: 40 ml/min

air flow: 450 ml/min

In FIG. 35, the catalytic conversion of cinnamaldehyde as a function of the reaction time is shown. Clearly, all tested catalysts are active in the hydrogenation of cinnamaldehyde.

In FIG. 36, the selectivities of these catalysts with respect to hydrocinnamaldehyde (HZAH), cinnamyl alcohol (ZAO) and hydrocinnamyl alcohol (HZAO) at a conversion of 5% are shown. It is noted that all catalysts show similar selectivities.

In FIG. 37, the selectivities of these catalysts with respect to hydrocinnamaldehyde (HZAH), cinnamyl alcohol (ZAO) and hydrocinnamyl alcohol (HZAO) after a reaction time of 300 min are shown. The respectively observed conversions after 300 min correlate with the findings from the TEM characterization: the largest particles have the smallest active surface and therefore the lowest conversion.

### Example 4.2 Dehydrogenation of Propane

The catalytic activities of the synthesized supported intermetallic compound of Example 2.1.6 (30 weight-% Cu₂NiSn/SiO₂) was tested in the dehydrogenation of propane. The time-on-stream experiment was carried out in a fixed-bed flow-type reactor at 650° C. The catalyst particle size was set to 255-355 micrometer by grounding and sieving. 250 mg of catalyst were tested in a mixture of flowing propane (3 ml/min) and nitrogen (27 ml/min) at atmospheric pressure. At regular time intervals, the product mixture was analyzed by gas chromatography. The conditions for the gas chromatographic analysis of the products from the dehydrogenation of propane were chosen as follows:

stationary phase: HP-Plot

length: 50 m

inner diameter: 530 micrometer

film thickness: 0.15 micrometer

oven temperature: 120° C.

inlet temperature: 200° C.

split ratio: 10:1

total flow rate: 81.6 ml/min

eluent: N₂

velocity: 56 cm/s

detector: flame ionization detector

makeup flow: 45 ml/min

hydrogen flow: 40 ml/min

air flow: 450 ml/min

It was found that after a reaction time of 21 h, the tested supported intermetallic compound shows a conversion of 42%, a selectivity with respect to propene of 40%, a selectivity with respect to ethene of 37.5%, a selectivity with respect to ethane of 2%, and a selectivity with respect to methane of 20.5%

### Example 4.3 Knoevenagel Condensation Reaction

The catalytic activities of the synthesized supported intermetallic compound of Examples 2.1.1 to 2.1.6 (30 weight-% Cu₂NiZ/SiO₂) were tested in the Knoevenagel condensation reaction.

Malononitrile (0.52 g, 8 mmol), benzaldehyde (0.84 g, 8 mmol), 20 ml toluene as a solvent and 0.2 g of 1,4-dichlorbenzene as internal standard were mixed in a 50 ml two-necked flask equipped with a reflux condenser. The mixture was heated in an oil bath and 0.4 g of the respective supported intermetallic compound were added when the maximum temperature of 80° C. was reached. At regular time intervals, the reaction mixture was analyzed by gas chromatography. The conditions for the gas chromatographic analysis of the products from the hydrogenation of cinnamaldehyde were chosen as follows:

stationary phase: CP-SIL 5 CB

length: 25 m

inner diameter: 250 micrometer

film thickness: 0.25 micrometer

oven temperature: start: 55° C. 1 min
40 K/min 250° C. 3 min

inlet temperature: 245° C.

split ratio: 50:1

total flow rate: 53.3 ml/min

eluent: He

velocity: 40 cm/s

detector: mass selective (MS) detector

In FIG. 38, the catalytic conversion of the benzaldehyde is shown as a function of the reaction time is shown. Clearly, all tested supported intermetallic compounds show a significantly higher activity than the support material alone.

### BRIEF DESCRIPTION OF THE FIGURES

FIG. 1: shows the X-ray diffraction pattern (copper K alpha radiation) of the supported intermetallic compound according to Example 1.1. On the x axis, the degree values (2theta) are shown, on the y axis, the intensity is shown.

FIG. 2: shows the X-ray diffraction pattern (copper K alpha radiation) of the supported intermetallic compound according to Example 1.2. On the x axis, the degree values (2theta) are shown, on the y axis, the intensity is shown.

FIG. 3: shows the X-ray diffraction pattern (copper K alpha radiation) of the supported intermetallic compound according to Example 1.3. On the x axis, the degree values (2theta) are shown, on the y axis, the intensity is shown.

FIG. 4: shows the X-ray diffraction pattern (copper K alpha radiation) of the supported intermetallic compound according to Example 1.4. On the x axis, the degree values (2theta) are shown, on the y axis, the intensity is shown.

FIG. 5: shows the X-ray diffraction pattern (copper K alpha radiation) of the supported intermetallic compound according to Example 1.5. On the x axis, the degree values (2theta) are shown, on the y axis, the intensity is shown.

FIG. 6: shows the X-ray diffraction pattern (copper K alpha radiation) of the supported intermetallic compound according to Example 1.6. On the x axis, the degree values (2theta) are shown, on the y axis, the intensity is shown.

FIG. 7: shows the X-ray diffraction pattern (copper K alpha radiation) of the supported intermetallic compound according to Example 1.7. On the x axis, the degree values (2theta) are shown, on the y axis, the intensity is shown.

FIG. 8: shows the X-ray diffraction pattern (copper K alpha radiation) of the supported intermetallic compound according to Example 1.8. On the x axis, the degree values (2theta) are shown, on the y axis, the intensity is shown.

FIG. 9: shows the X-ray diffraction pattern (copper K alpha radiation) of the supported intermetallic compound according to Example 2.1.1. On the x axis, the degree values (2theta) are shown, on the y axis, the intensity is shown.

FIG. 10: shows the X-ray diffraction pattern (copper K alpha radiation) of the supported intermetallic compound according to Example 2.1.2. On the x axis, the degree values (2theta) are shown, on the y axis, the intensity is shown.

FIG. 11: shows the X-ray diffraction pattern (copper K alpha radiation) of the supported intermetallic compound according to Example 2.1.3. On the x axis, the degree values (2theta) are shown, on the y axis, the intensity is shown.

FIG. 12: shows the X-ray diffraction pattern (copper K alpha radiation) of the supported intermetallic compound according to Example 2.1.4. On the x axis, the degree values (2theta) are shown, on the y axis, the intensity is shown.

FIG. 13: shows the X-ray diffraction pattern (copper K alpha radiation) of the supported intermetallic compound according to Example 2.1.5. On the x axis, the degree values (2theta) are shown, on the y axis, the intensity is shown.

FIG. 14: shows the X-ray diffraction pattern (copper K alpha radiation) of the supported intermetallic compound according to Example 2.1.6. On the x axis, the degree values (2theta) are shown, on the y axis, the intensity is shown.

FIG. 15: shows the X-ray diffraction pattern (copper K alpha radiation) of the supported intermetallic compound according to Example 2.2.1. On the x axis, the degree values (2theta) are shown, on the y axis, the intensity is shown.

FIG. 16: shows the X-ray diffraction pattern (copper K alpha radiation) of the supported intermetallic compound according to Example 2.2.2. On the x axis, the degree values (2theta) are shown, on the y axis, the intensity is shown.

FIG. 17: shows the X-ray diffraction pattern (copper K alpha radiation) of the supported intermetallic compound according to Example 2.2.3. On the x axis, the degree values (2theta) are shown, on the y axis, the intensity is shown.

FIG. 18: shows the X-ray diffraction pattern (copper K alpha radiation) of the supported intermetallic compound according to Example 2.2.4. On the x axis, the degree values (2theta) are shown, on the y axis, the intensity is shown.

FIG. 19: shows the X-ray diffraction pattern (copper K alpha radiation) of the supported intermetallic compound according to Example 2.2.5. On the x axis, the degree values (2theta) are shown, on the y axis, the intensity is shown.

FIG. 20: shows the X-ray diffraction pattern (copper K alpha radiation) of the supported intermetallic compound according to Example 2.2.6. On the x axis, the degree values (2theta) are shown, on the y axis, the intensity is shown.

FIG. 21: shows the X-ray diffraction pattern (copper K alpha radiation) of the supported intermetallic compound according to Example 2.3.1. On the x axis, the degree values (2theta) are shown, on the y axis, the intensity is shown.

FIG. 22: shows the X-ray diffraction pattern (copper K alpha radiation) of the supported intermetallic compound according to Example 2.3.2. On the x axis, the degree values (2theta) are shown, on the y axis, the intensity is shown.

FIG. 23: shows the X-ray diffraction pattern (copper K alpha radiation) of the supported intermetallic compound according to Example 2.3.3. On the x axis, the degree values (2theta) are shown, on the y axis, the intensity is shown.

FIG. 24: shows the X-ray diffraction pattern (copper K alpha radiation) of the supported intermetallic compound according to Example 2.3.4. On the x axis, the degree values (2theta) are shown, on the y axis, the intensity is shown.

FIG. 25: shows the X-ray diffraction pattern (copper K alpha radiation) of the supported intermetallic compound according to Example 2.3.5. On the x axis, the degree values (2theta) are shown, on the y axis, the intensity is shown.

FIG. 26: shows the X-ray diffraction pattern (copper K alpha radiation) of the supported intermetallic compound according to Example 2.3.6. On the x axis, the degree values (2theta) are shown, on the y axis, the intensity is shown.

FIG. 27: shows the X-ray diffraction pattern (copper K alpha radiation) of the supported intermetallic compound according to Example 2.3.7. On the x axis, the degree values (2theta) are shown, on the y axis, the intensity is shown.

FIG. 28: shows the crystallite size (in nm) of the Heusler compounds of Examples 2.3.1 to 2.3.4 determined according to Reference Example 1.2 as a function of the metal content (in weight-%) of the respective compounds.

FIG. 29: shows 2 TEM images of the particles of the intermetallic compound of Example 2.1.6. In the left image, the scale bar in the right lower corner represents 1 micrometer. In the right image, the scale bar in the right lower corner represents 200 nm.

FIG. 30: shows 2 TEM images of the particles of the intermetallic compound of Example 2.3.1. In the left image, the scale bar in the right lower corner represents 1 micrometer. In the right image, the scale bar in the right lower corner represents 1 micrometer.

FIG. 31: shows 2 TEM images of the particles of the intermetallic compound of Example 2.3.4. In the left image, the scale bar in the right lower corner represents 1 micrometer. In the right image, the scale bar in the right lower corner represents 200 nm.

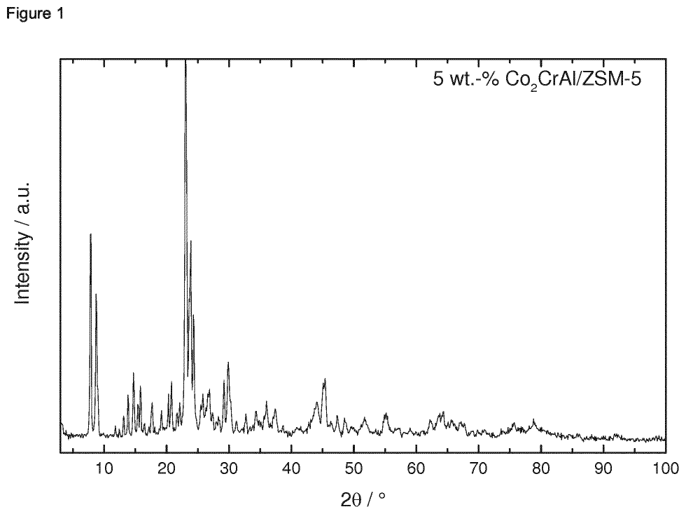

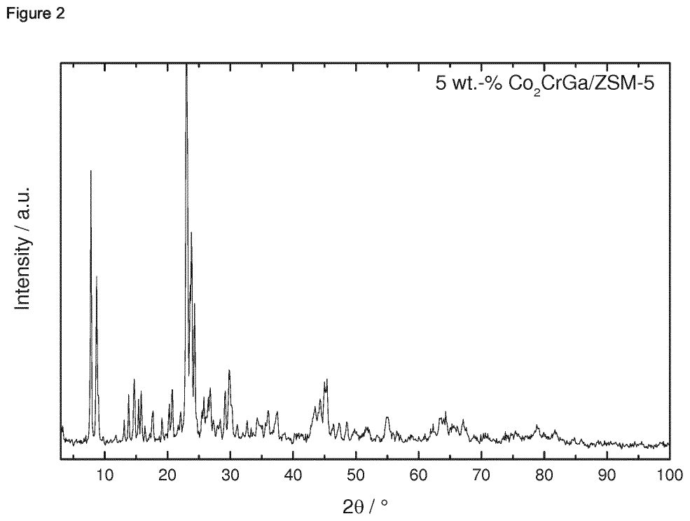

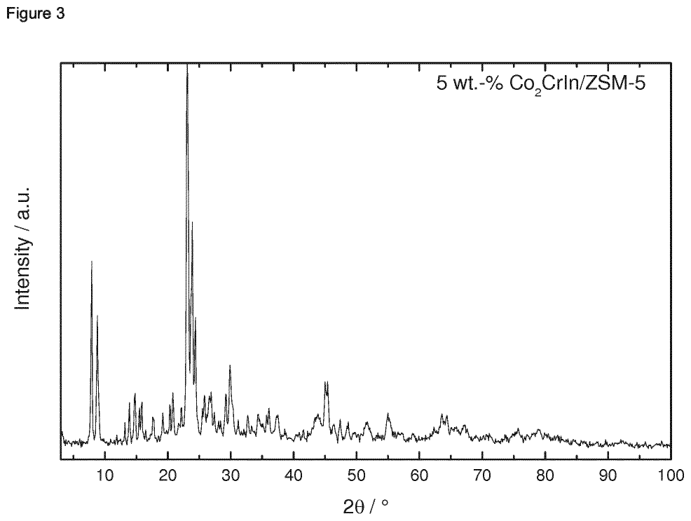

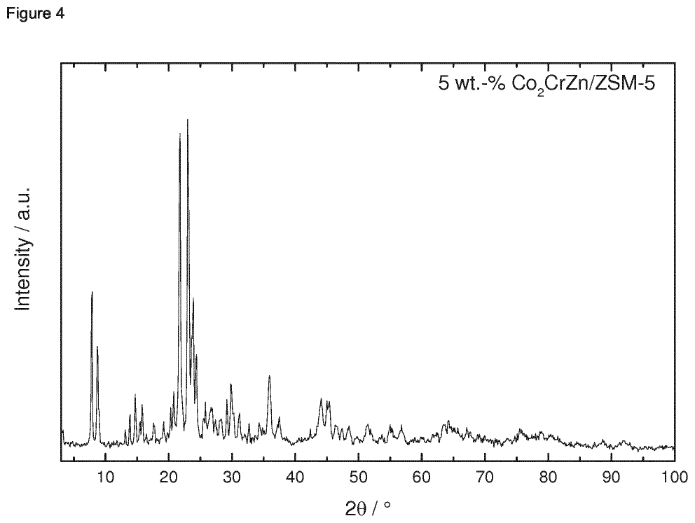

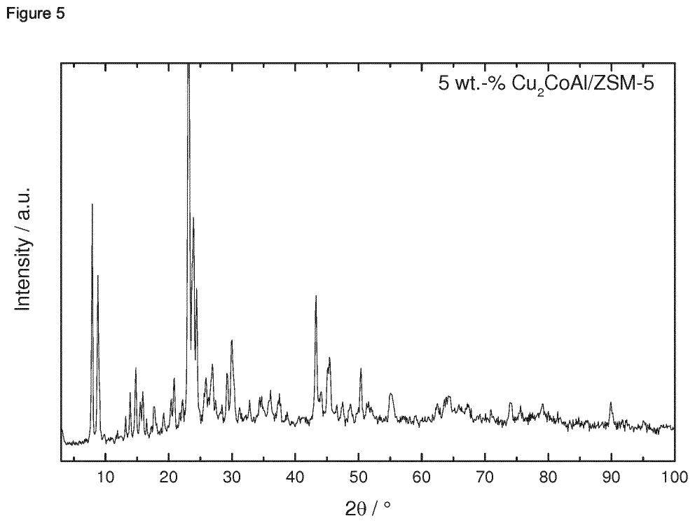

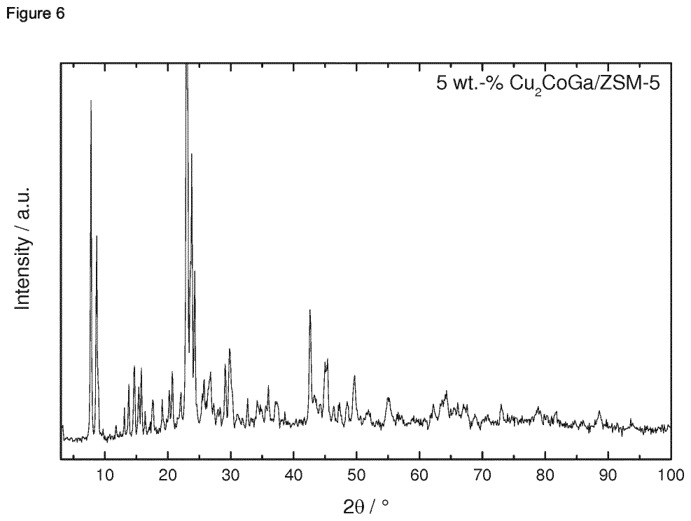

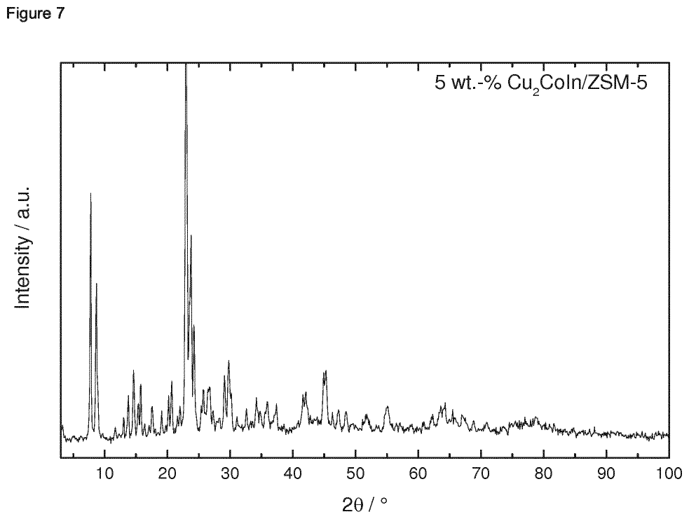

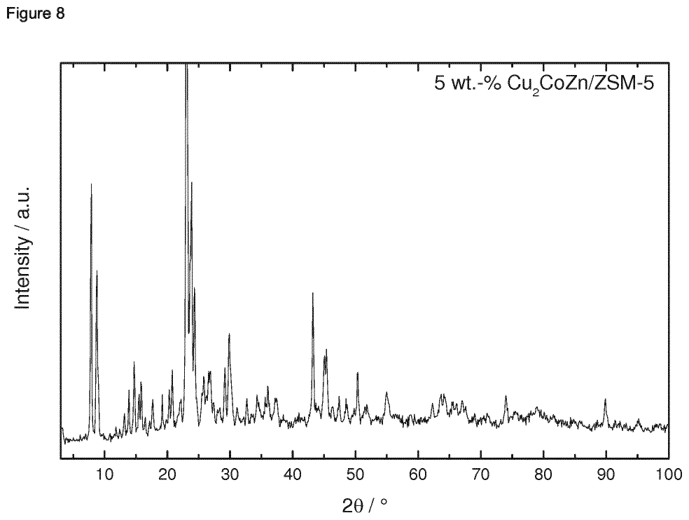

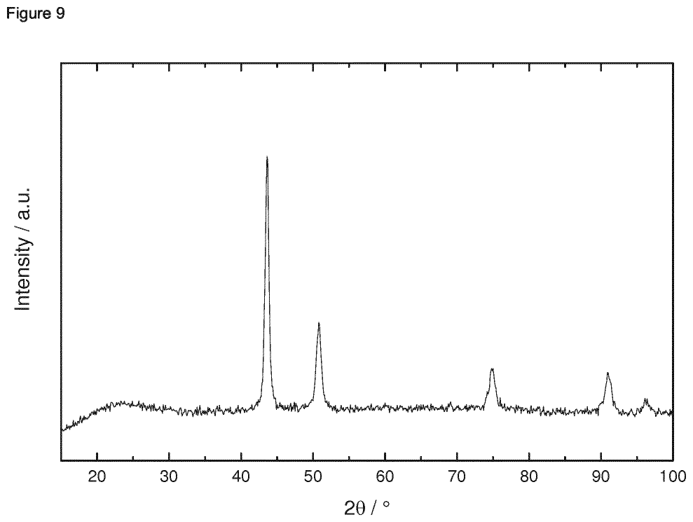

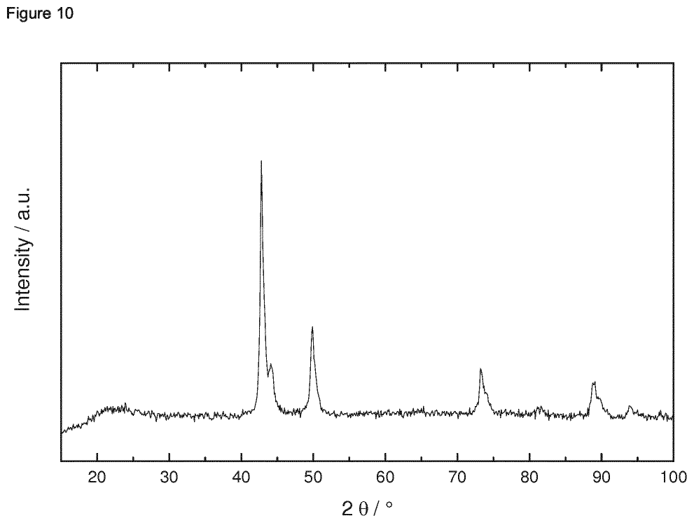

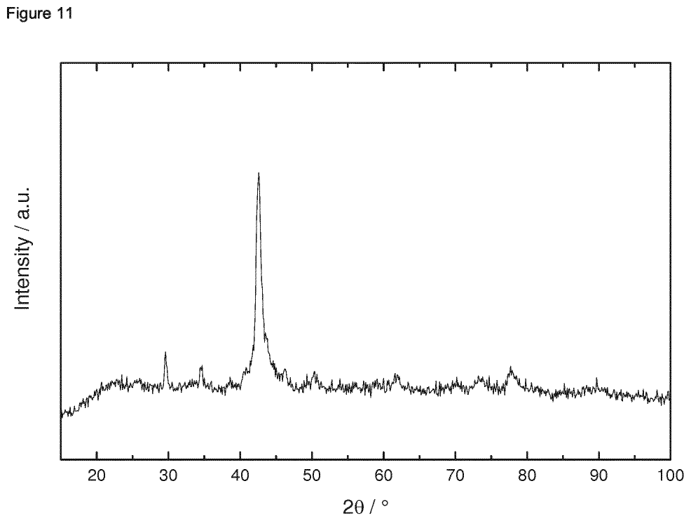

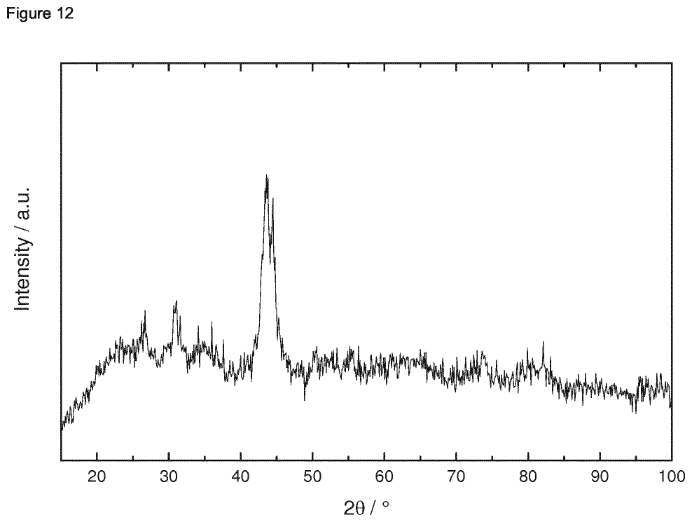

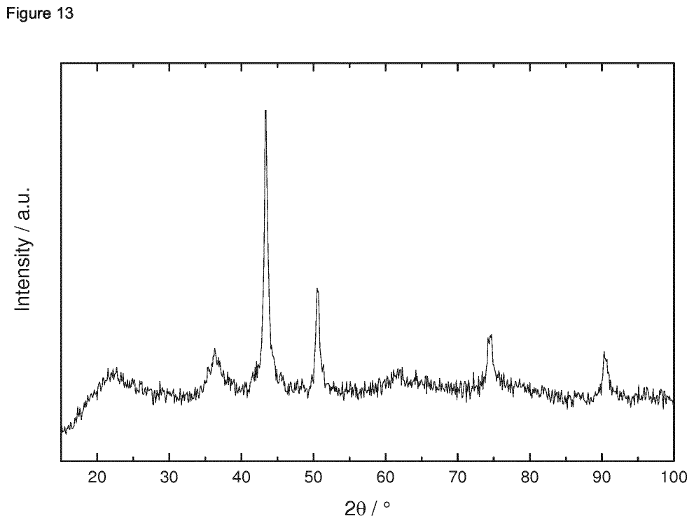

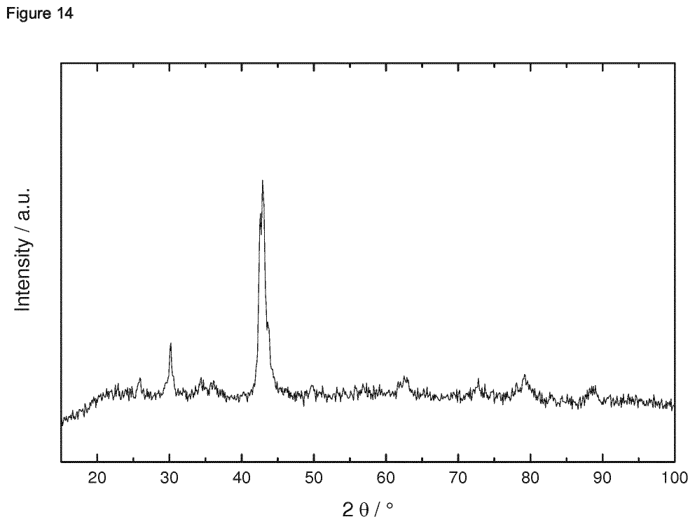

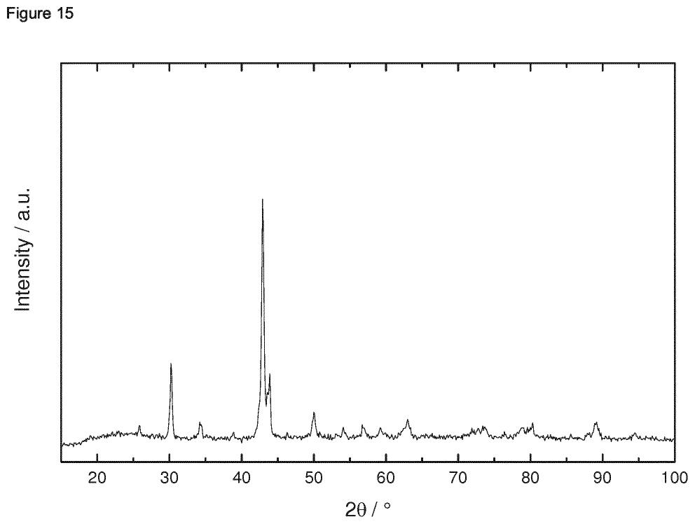

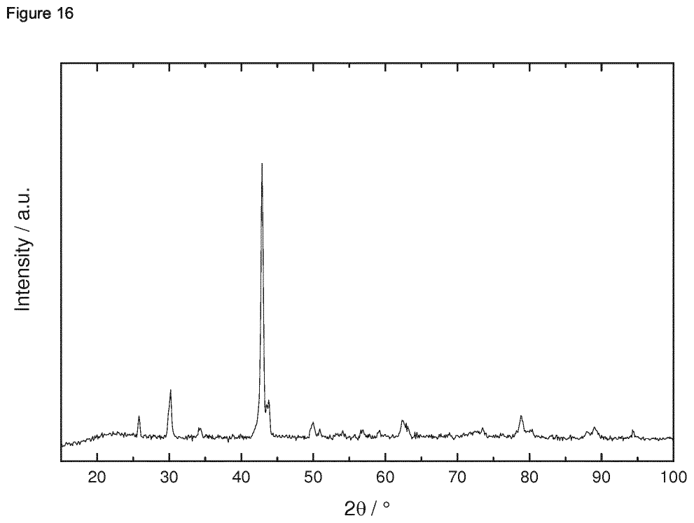

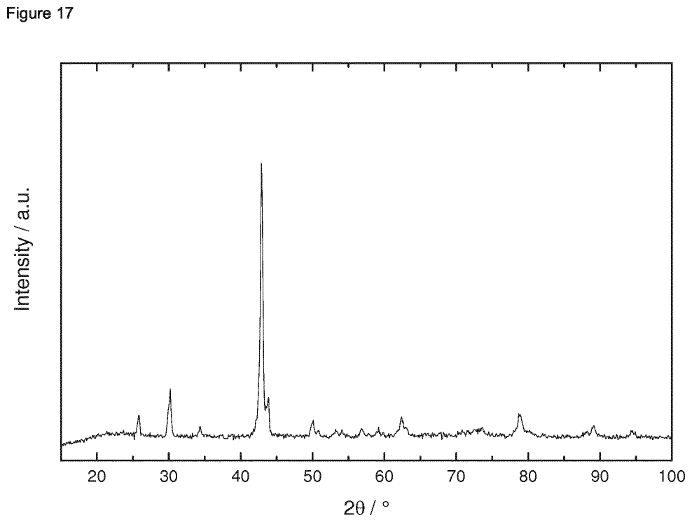

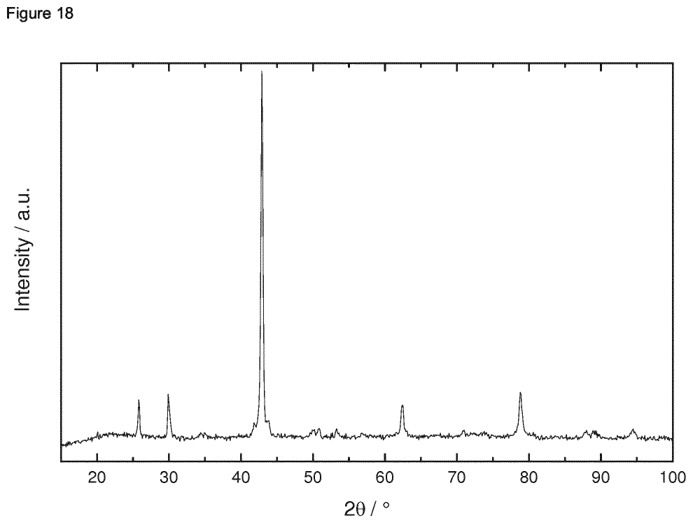

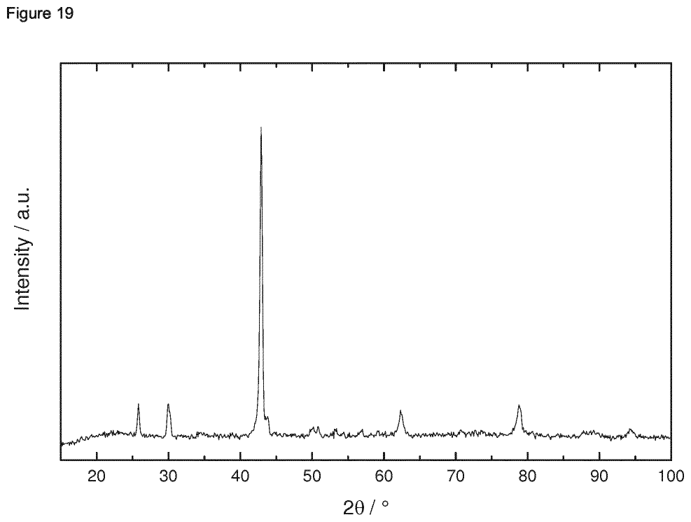

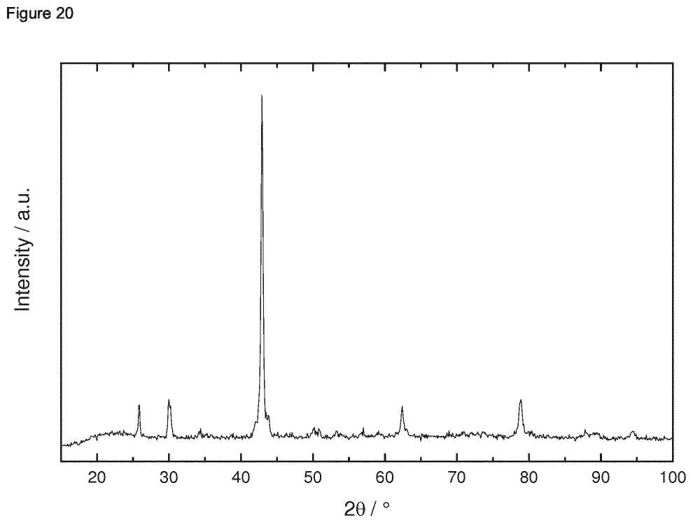

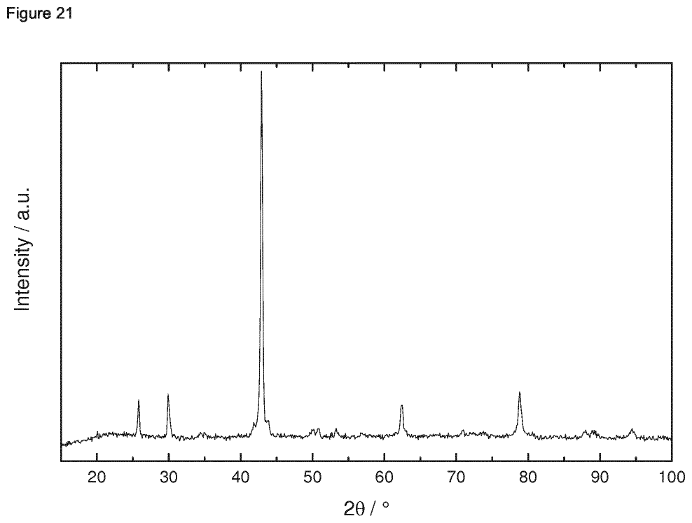

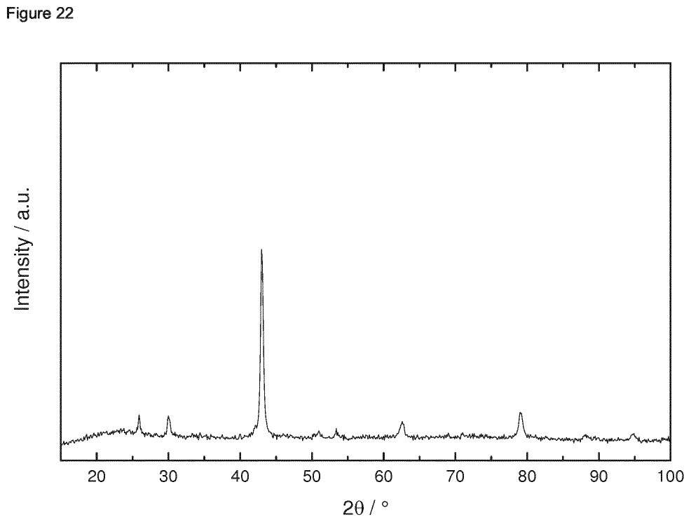

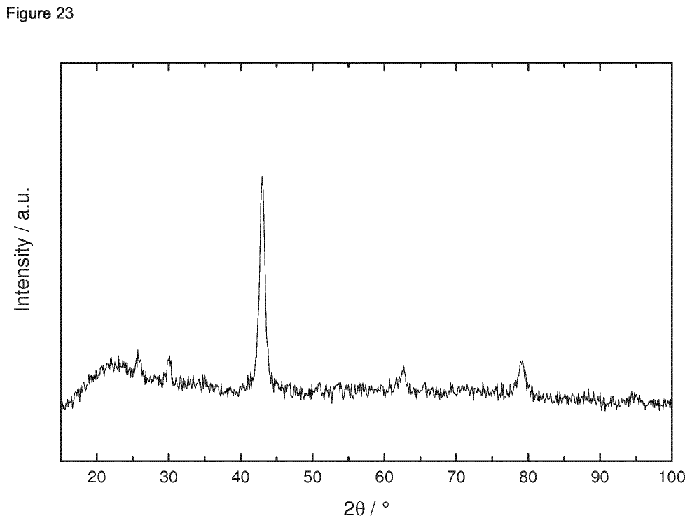

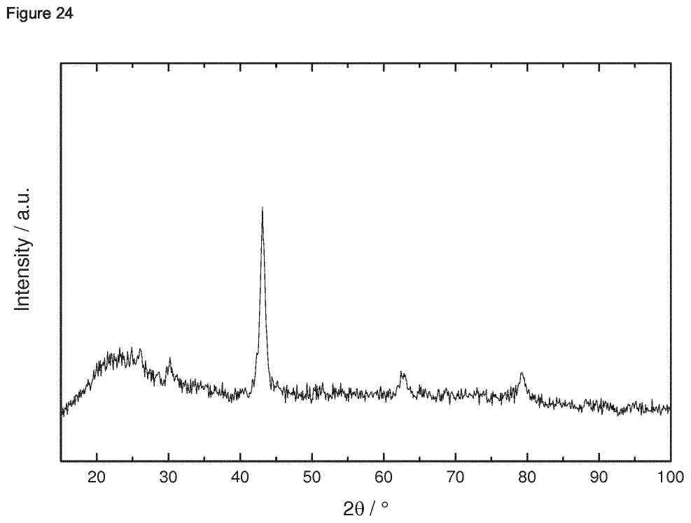

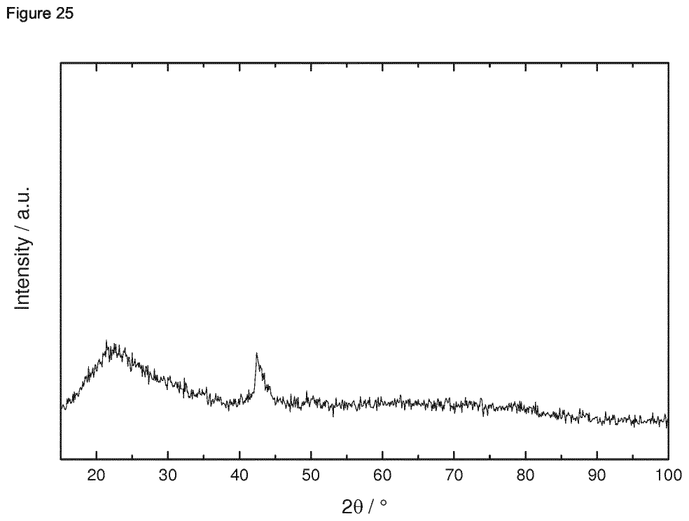

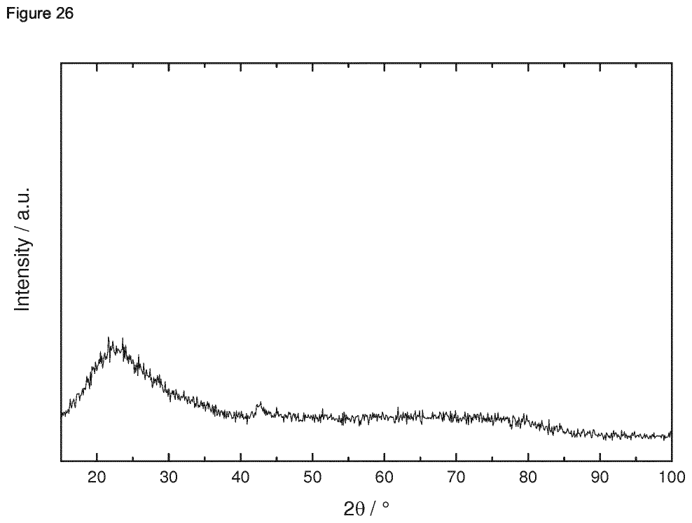

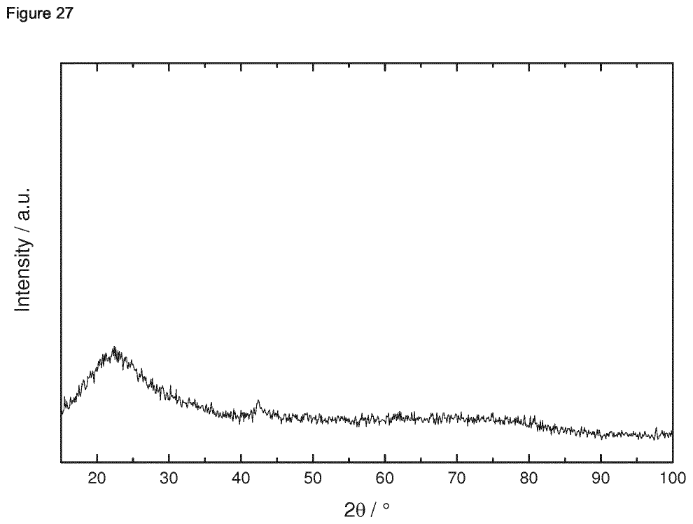

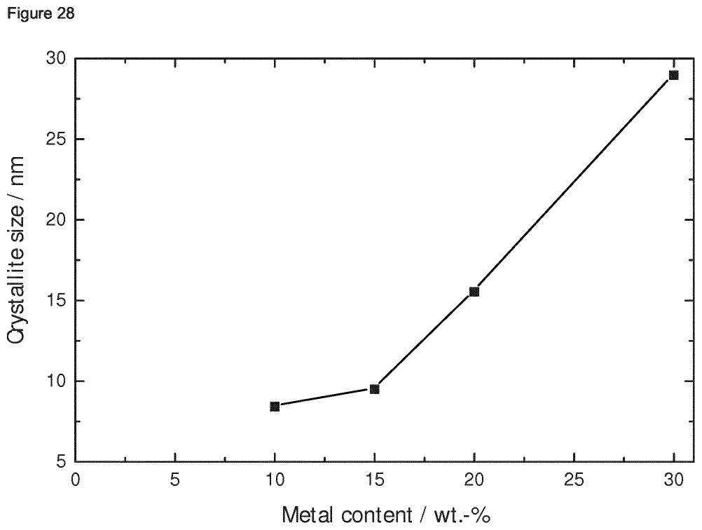

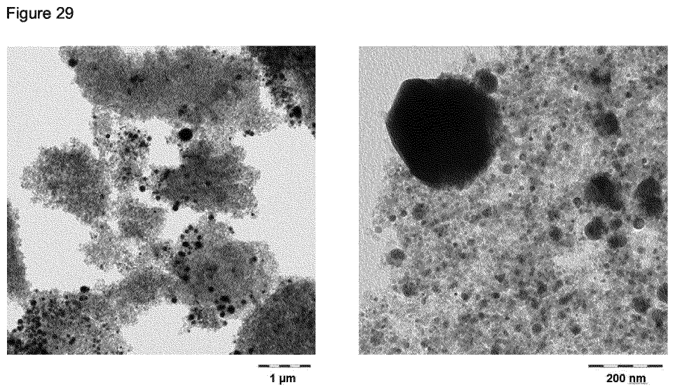

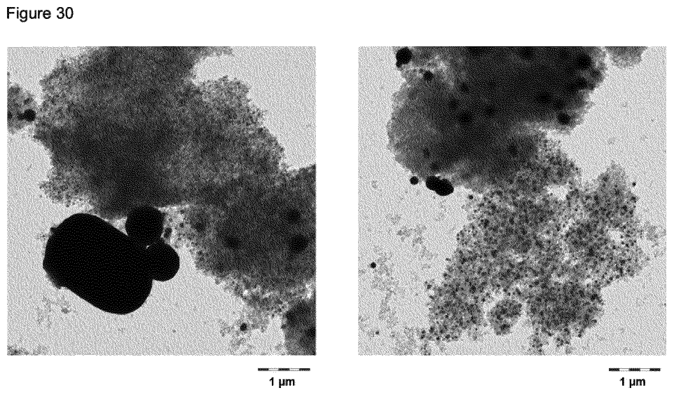

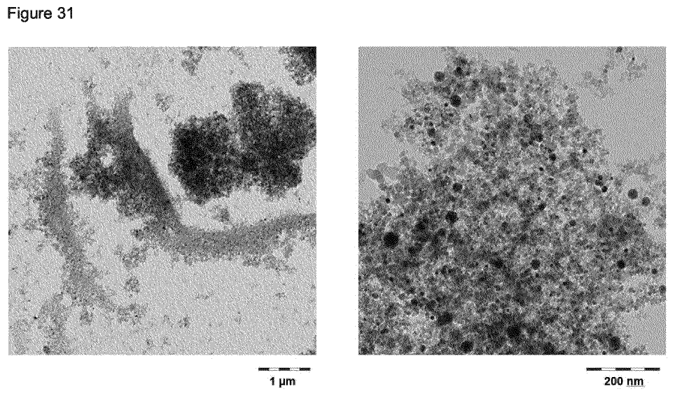

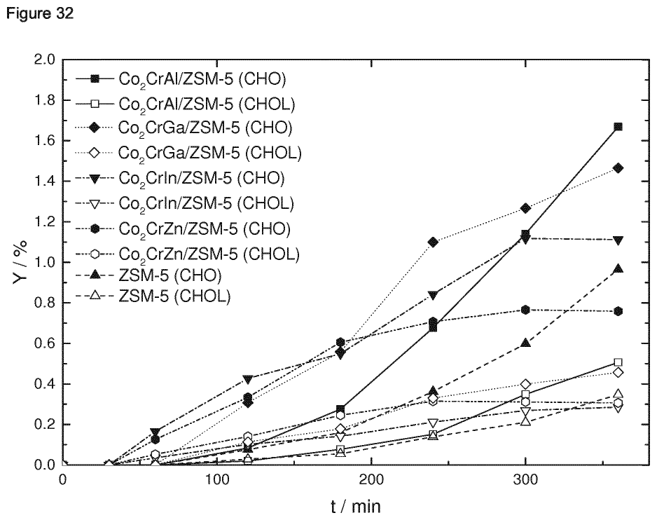

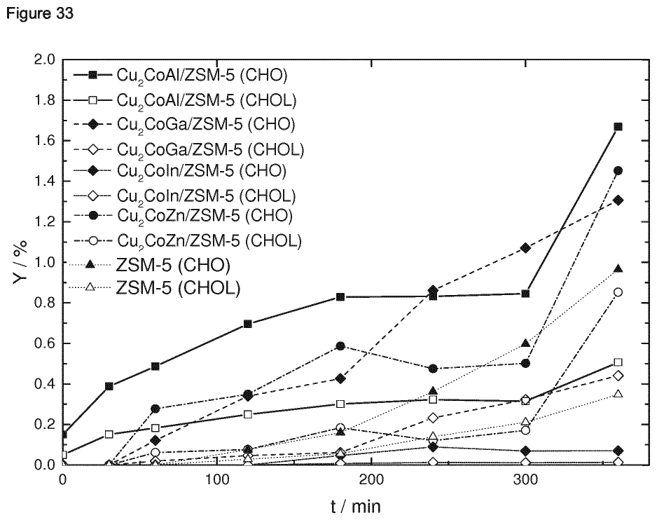

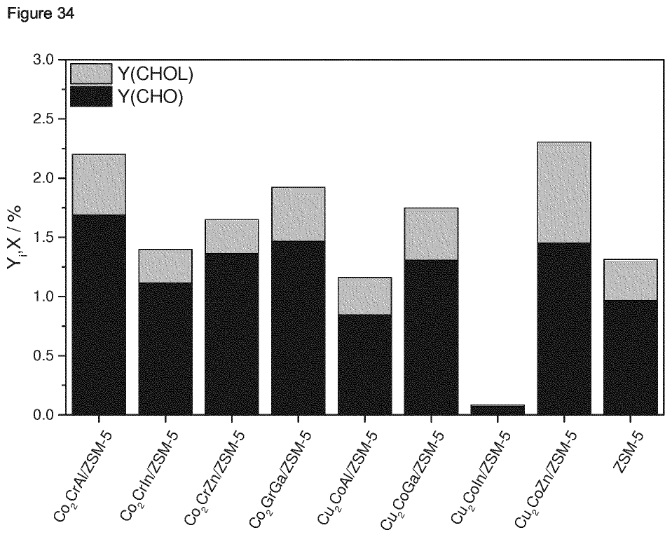

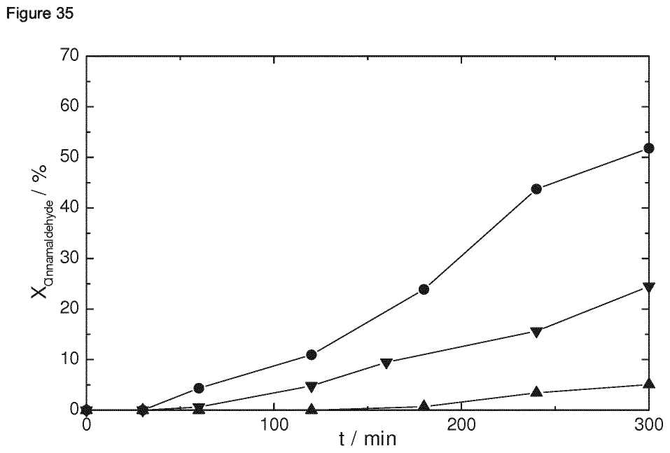

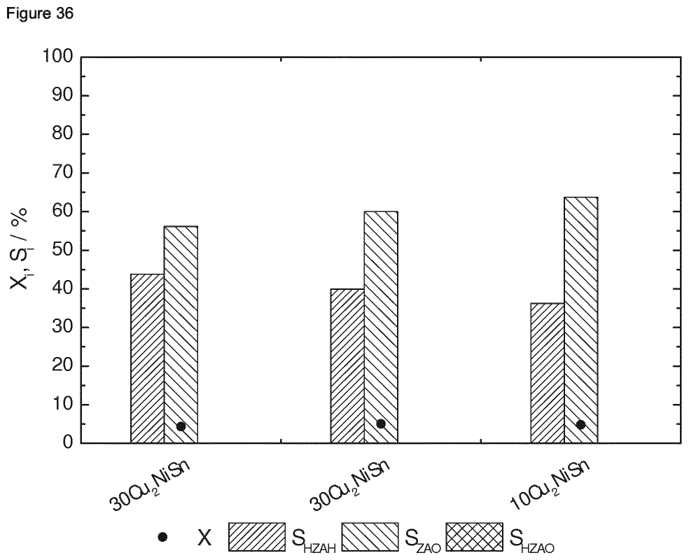

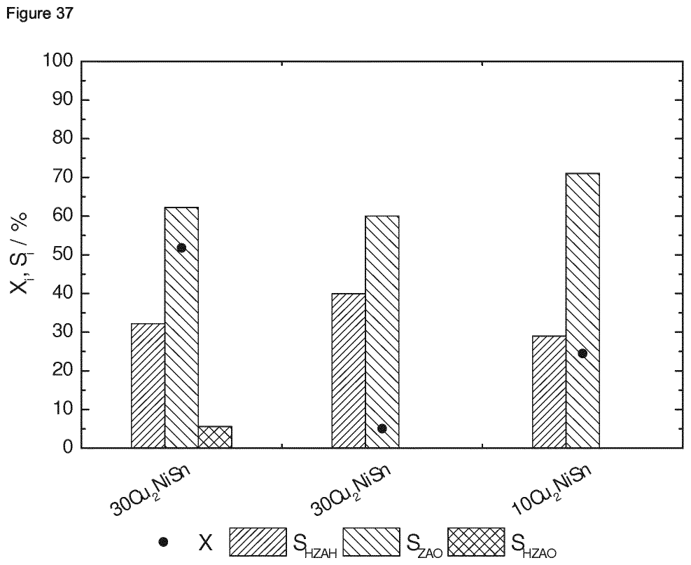

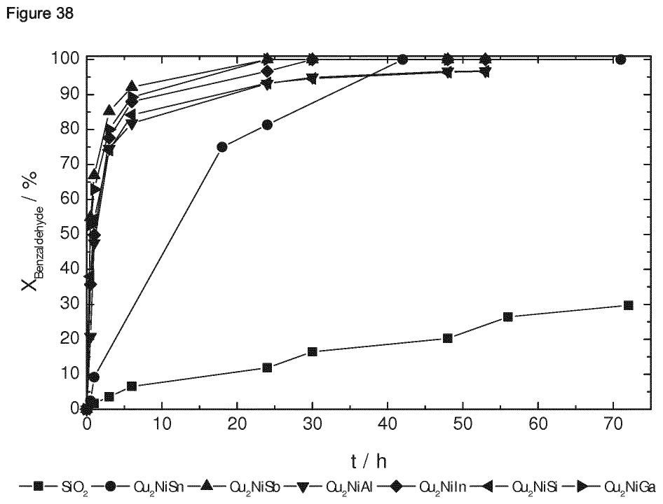

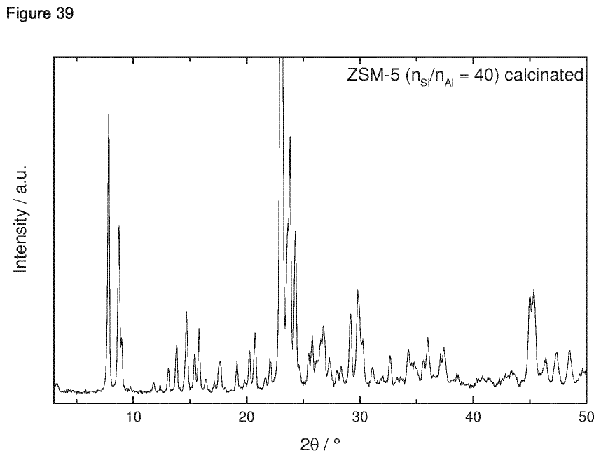

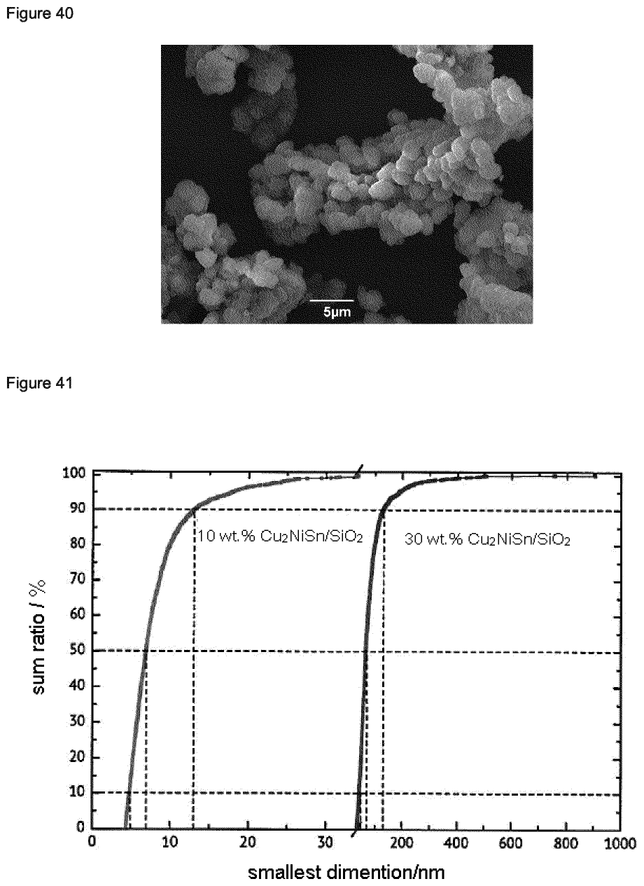

FIG. 32: shows the yields (Y/%) of cyclohexanone (CHO) and cyclohexanole (CHOL) obtained when using the intermetallic compounds of Examples 1.1 to 1.4 (Co₂CrAl/ZSM-5, Co₂CrGa/ZSM-5, Co₂CrIn/ZSM-5, Co₂CrZn/ZSM-5), as described in Example 3, as a function of the reaction time (t/min), as follows:
filled square: Co₂CrAl/ZSM-5, yield (CHO)
empty square: Co₂CrAl/ZSM-5, yield (CHOL)
filled diamond: Co₂CrGa/ZSM-5, yield (CHO)
empty diamond: Co₂CrGa/ZSM-5, yield (CHOL)
filled triangle tip down: Co₂CrIn/ZSM-5, yield (CHO)
empty triangle tip down: Co₂CrIn/ZSM-5, yield (CHOL)
filled circle: Co₂CrZn/ZSM-5, yield (CHO)
empty circle: Co₂CrZn/ZSM-5, yield (CHOL)
filled triangle tip up: ZSM-5, yield (CHO)
empty triangle tip up: ZSM-5, yield (CHOL)

FIG. 33: shows the yields (Y/%) of cyclohexanone (CHO) and cyclohexanole (CHOL) obtained when using the intermetallic compounds of Examples 1.5 to 1.8 (Cu₂CoAl/ZSM-5, Cu₂CoGa/ZSM-5, Cu₂CoIn/ZSM-5, Cu₂CoZn/ZSM-5), as described in Example 3, as a function of the reaction time (t/min), as follows:
filled square: Cu₂CoAl/ZSM-5, yield (CHO)
empty square: Cu₂CoAl/ZSM-5, yield (CHOL)
filled diamond, dashed line: Cu₂CoGa/ZSM-5, yield (CHO)
empty diamond, dashed line: Cu₂CoGa/ZSM-5, yield (CHOL)
filled diamond, dotted line: Cu₂CoIn/ZSM-5, yield (CHO)
empty diamond, dotted line: Cu₂CoIn/ZSM-5, yield (CHOL)
filled circle: Cu₂CoZn/ZSM-5, yield (CHO)
empty circle: Cu₂CoZn/ZSM-5, yield (CHOL)
filled triangle tip up: ZSM-5, yield (CHO)
empty triangle tip up: ZSM-5, yield (CHOL)

FIG. 34: shows the results of FIGS. 33 and 34 in condensed form. For each catalyst (from left to right: Co₂CrAl/ZSM-5, Co₂CrIn/ZSM-5, Co₂CrZn/ZSM-5, Co₂CrGa/ZSM-5, Cu₂CoAl/ZSM-5, Cu₂CoGa/ZSM-5, Cu₂CoIn/ZSM-5, Cu₂CoZn/ZSM-5, ZSM-5), the yields/% are shown in a separate column wherein the lower part of the column shows yield (CHO), the upper part of the column shows yield (CHOL) FIG. 35: shows the catalytic conversion of cinnamaldehyde in the hydrogenation reaction according to Example 4.1 using the supported intermetallic compounds of
Example 2.1.6 (30 weight-% Cu₂NiSn/SiO₂)—symbol: black filled circle
Example 2.3.1 (30 weight-% Cu₂NiSn/SiO₂)—symbol: black filled triangle tip up
Example 2.3.4 (30 weight-% Cu₂NiSn/SiO₂)—symbol: black filled triangle tip down

FIG. 36: shows the conversion (X; symbol: bullet point (filled circle)) and the selectivities (S) of the three tested supported intermetallic compounds according to Example 4.1. For each catalyst, the selectivity with respect to HZAH and the selectivity with respect to ZAO are shown from left to right in individual columns. The two columns on the left refer to the selectivities the supported intermetallic compound of Example 2.1.6, the two columns in the middle refer to the selectivities the supported intermetallic compound of Example 2.3.1, the two columns on the right refer to the selectivities the supported intermetallic compound of Example 2.3.4. The selectivity with respect to HZAO was 0% for all tested supported intermetallic compounds.

FIG. 37: shows the conversion (X; symbol: bullet point (filled circle)) and the selectivities (S) of the three tested supported intermetallic compounds according to Example 4.1. For each catalyst, the selectivity with respect to HZAH, the selectivity with respect to ZAO and the selectivity with respect to HZAO are shown from left to right in individual columns. The three columns on the left refer to the selectivities the supported intermetallic compound of Example 2.1.6, the two columns in the middle refer to the selectivities the supported intermetallic compound of Example 2.3.1 (selectivity with respect to HZAO=0), the two columns on the right refer to the selectivities the supported intermetallic compound of Example 2.3.4 (selectivity with respect to HZAO=0).

FIG. 38: shows the conversion (X) of the six tested supported intermetallic compounds according to Examples 2.1.1 to 2.1.6. The symbols from left to right:
filled square (SiO₂ support)
filled circle (30 weight-% Cu₂NiSn/SiO₂)
filled triangle tip up (30 weight-% Cu₂NiSb/SiO₂)
filled triangle tip down (30 weight-% Cu₂NiAl/SiO₂)
filled diamond (30 weight-% Cu₂NiIn/SiO₂)
filled triangle tip left (30 weight-% Cu₂NiSi/SiO₂)
filled triangle tip right (30 weight-% Cu₂NiGa/SiO₂)

FIG. 39: shows the X-ray diffraction pattern (copper K alpha radiation) of the zeolitic material (ZSM-5) used as a support material. On the x axis, the degree values (2theta) are shown, on the y axis, the intensity is shown.

FIG. 40: shows an SEM image of the zeolitic material (ZSM-5) used as a support material. The scale bar in the middle represents 5 micrometer.

FIG. 41: shows sum ratio distribution of the Heusler compounds of Examples 2.3.4 and Example 2.3.1 in % as a function of the smallest crystalline size in nm as determined according to Reference Example 1.1. As one can see, FIG. 41 is divided into two parts as highlighted by the two different scales therein along the x axis for the smallest dimension (see break in scale indicated by “/” in the x axis).

### CITED LITERATURE

WO 2017/029165 A
Hedin et al., Z. physik. Chem. B30 (1935), pages 280-288
Kojima et al., ACS Omega 2 (2017) pages 147-153
Senanayake et al. “Exploring Heusler alloys as catalysts for ammonia dissociation”, August 2016, ISBN: 978-1-369-00770-1
Okamura et al. “Structural, magnetic, and transport properties of full-Heusler alloy Co₂(Cr₁₋ₓFeₓ)Al thin films” J. Appl. Phys. vol. 96, no. 11, 1 Dec. 2004, pages 6561-6564—Kelekar et al. “Epitaxial growth of the Heusler alloy Co₂Crₐ₋ₓFeₓAl” J. Appl. Phys. Vol. 96, no 1, 1 Jul. 2004, pages 540-543
Ko et al. “Half-metallic Fe₂CrSi and non-metallic Cu₂CrAl Heusler alloys for currentperpendicular-to-plane giant magneto-resistance: First principle and experimental study” J. Appl. Phys. Vol 109, no. 7, 17 Mar. 2011, pages 7B1031-7B1033

1: A composition comprising a ternary intermetallic compound X₂YZ, wherein
X, Y, and Z are different from one another;
X being selected from the group consisting of Mn, Fe, Co, Ni, Cu, and Pd;
Y being selected from the group consisting of Cr, Co, and Ni; and
Z being selected from the group consisting of Al, Si, Ga, Ge, In, Sn, Zn, and Sb;
wherein the ternary intermetallic compound is supported on a porous oxidic support material.

2: The composition of claim 1, wherein the porous oxidic support material comprises one or more selected from the group consisting of:
silica,
alumina,
titania,
zirconia, and
a mixed oxide of one or more selected from the group consisting of Si, Al, Ti, and Zr.

3: The composition of claim 1, wherein X or Y is Co, and wherein Z is selected from the group consisting of Al, Ga, In, and Zn.

4: The composition of claim 1, wherein the porous oxidic support material comprises a mixed oxide of Si and Al.

5: The composition of claim 1, wherein in the composition, the weight ratio of the ternary intermetallic compound relative to the porous oxidic compound is in the range of from 0.5:99.5 to 30:70.

6: The composition of claim 1, wherein Y is Ni, and wherein Z is Al, Si, Ga, In, Sn, or Sb.

7: The composition of claim 6, wherein the porous oxidic support material comprises silica.

8: The composition of claim 6, wherein in the composition, the weight ratio of the ternary intermetallic compound relative to the porous oxidic support is in the range of from 1:99.5 to 70:30.

9: The composition of claim 6, having a BET specific surface area in the range of from 150 to 400 m²/g.

10: The composition of claim 1, wherein at least 99 weight-% of the composition consists of the ternary intermetallic compound and the porous oxidic support material.

11: The composition of claim 1, wherein the intermetallic compound is a Heusler phase.

12: A process for preparing the composition of claim 1, comprising
(i) preparing a liquid mixture comprising a source of X, a source of Y, a source of Z, and a source of the porous oxidic support material;
(ii) removing the liquid phase from the mixture prepared in (i); and
(iii) heating the mixture obtained from (ii) in a reducing atmosphere, thereby obtaining the intermetallic compound supported on the porous oxidic support material.

13: The process of claim 12, wherein the source of X is selected from the group consisting of salts of X, wherein the salts of X are selected from the group consisting of acetates, acetylacetonates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof; wherein the source of Y is selected from the group consisting of salts of Y, wherein the salts of Y are selected from the group consisting of acetates, acetylacetonates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof; wherein the source of Z is selected from the group consisting of salts of Z, wherein the salts of Z are selected from the group consisting of C1-C4 alkoxides, acetates, nitrates, nitrites, sulfates, hydrogensulfates, dihydrogensulfates, sulfites, hydrogensulfites, phosphates, hydrogenphosphates, dihydrogenphosphates, halides, cyanides, cyanates, isocyanates, and mixtures of two or more thereof; and wherein the source of the porous oxidic support material comprises one or more selected from the group consisting of silica, alumina, titania, zirconia, and a mixed oxide of one or more Si, Al, Ti, and Zr.

14: The process of claim 12, wherein (i) comprises
(i.1) preparing a liquid mixture comprising a source of X, a source of Y, a source of Z, and a solvent; and
(i.2) admixing the source of the porous oxidic support material with the mixture prepared in (i.1);
wherein the solvent according to (i. 1) is a polar solvent; and
wherein the solvent according to (i.2) is a polar solvent.

15: The process of claim 12, wherein removing the liquid phase from the mixture according to (ii) comprises heating the mixture prepared in (i).

16: The process of claim 12, wherein the reducing atmosphere according to (iii) comprises hydrogen.

17: The process of claim 12, further comprising
(iv) cooling the intermetallic compound supported on the porous oxidic material, obtained from (iii).

18-19. (canceled)
